(19) United States
(12) Patent Application Publication
(10) Pub. No.: US 2022/0101147 A1
(43) Pub. Date: Mar. 31, 2022

(54) SYSTEM AND METHOD FOR PREDICTING TRAIT INFORMATION OF INDIVIDUALS

(71) Applicant: OSAKA UNIVERSITY, Osaka (JP)

(72) Inventors: Masamitsu KONNO, Osaka (JP); Hideshi ISHII, Osaka (JP); Masaki MORI, Osaka (JP); Ayumu ASAI, Osaka (JP); Jun KOSEKI, Osaka (JP)

(21) Appl. No.: 17/418,168

(22) PCT Filed: Dec. 27, 2019
(86) PCT No.: PCT/JP2019/051564

§ 371 Date: Jun. 24, 2021

(30) Foreign Application Priority Data
Dec. 28, 2018 (JP) 2018-247959

(51) Int. Cl. G06N 3/12; G06N 20/00

(52) U.S. Cl. CPC G06N 3/126; G06N 20/00

(57) ABSTRACT

The present disclosure relates to predicting trait information from the genetic information of individuals, and generating a model therefor. Learning is performed using a plurality of types of genetic information from a plurality of individuals, and a model for predicting trait information is generated. For said learning, it is possible to create images of the genetic information, and provide the same to said learning. The images in the present disclosure can store both sequence information and expression information. Moreover, the layout of genetic factors in the images can be optimized. Said learning can be performed as split learning, and the data after said split learning can be consolidated.

### TECHNICAL FIELD

The present disclosure relates to the field of data analysis. More specifically, the present disclosure relates to a technology for predicting trait information on an individual from data of genetic information on the individual.

### BACKGROUND ART

The recent advancement in measurement technologies has enabled the collection of a large amount of more diverse genetic information on an individual. For example, nucleic acid sequences including genomic sequences, information on gene expression, information on expression of a non-coding nucleic acid, information on epigenetic modifications, and the like can be collected. With the premise that traits of an individual are defined based on genetic information, traits of an individual should be, in principle, predictable in advance if genetic information can be comprehensively acquired. However, genetic information on an individual contains a very large amount of information, and contribution thereof to traits is affected by various factors in a complex manner. Thus, such a prediction is still challenging.

### SUMMARY OF INVENTION

### Solution to Problem

In one embodiment of the present disclosure, a system for predicting trait information on an individual, or a method, program, and recording medium using the same is provided. Such an embodiment of the present disclosure is intended to enable prediction of trait information on an individual from genetic information on the individual by learning from trait information on a plurality of individuals, and display of a prediction result. For example, the relationship between genetic information and trait information can be learned from genetic information on a plurality of individuals and trait information on the plurality of individuals. In particular, the embodiment can learn using a plurality of pieces of genetic information (e.g., sequence information (e.g., mutation information), expression information, modification information (e.g., methylation information), and the like on a genetic factor) as the genetic information, predict trait information based on the learning, and display the result thereof.

In one embodiment of the present disclosure, learning can comprise forming an image of genetic information on a plurality of individuals for learning. Such image formation can be performed, for example, as described in detail elsewhere herein. Data formed into an image can have a data format that is described in detail elsewhere herein. This can maximize the performance of artificial intelligence when learning a large amount of data associated with a plurality of types of genetic information simultaneously by artificial intelligence.

In one embodiment of the present disclosure, learning can be performed so that genetic information is divided, the relationships between partial genetic information and trait information is learned, then the relationships between a plurality of pieces of partial genetic information and trait information are integrated to learn the relationship between genetic information and trait information. This can overcome the limitation with respect to the amount of data in genetic information.

Examples of the present disclosure include the following items.

### [Item A1]

A system for predicting trait information on an individual, comprising:

a storage unit for storing genetic information on a plurality of individuals and trait information on the plurality of individuals, the genetic information containing at least two types of information;

a learning unit configured to learn a relationship between genetic information and trait information from the genetic information on the plurality of individuals and the trait information on the plurality of individuals; and a calculation unit for predicting trait information on an individual from genetic information on the individual based on the relationship between the genetic information and the trait information.

### [Item A2]

The system of the preceding item, wherein the learning unit is configured to learning after forming an image of the genetic information on the plurality of individuals. [Item A3]

The system of any of the preceding items, wherein the learning unit is configured to divide the genetic information on the plurality of individuals, learn relationships between partial genetic information and trait information, and integrate relationships between a plurality of pieces of partial genetic information and trait information to learn the relationship between the genetic information and the trait information.

### [Item A4]

The system of any of the preceding items, wherein the genetic information is selected from the group consisting of sequence information (e.g., mutation information), expression information, and modification information (e.g., methylation information) on a genetic factor.

### [Item A5]

The system of any of the preceding items, wherein the formation of an image of the genetic information on the plurality of individuals is configured to be performed by the image formation method of any of item B.

### [Item A6]

The system of any of the preceding items, wherein the learning unit is configured to use data with the data structure of any of item C in learning.

### [Item A7]

The system of any of the preceding items, wherein the learning unit is configured to learn the relationship between the genetic information and the trait information by the method of any of item D.

### [Item A8]

The system of any of the preceding items, comprising an analysis unit for analyzing diagnosis of the individual and/or treatment or prophylaxis on the individual from the trait information predicted in the calculation unit.

### [Item A9]

The system of any of the preceding items, further comprising a display unit for displaying the trait information predicted in the calculation unit.

### [Item A1-1]

A method for predicting trait information on an individual, comprising:

an information providing step for providing genetic information on a plurality of individuals and trait information on the plurality of individuals, the genetic information containing at least two types of information;

a learning step for learning a relationship between genetic information and trait information from the genetic information on the plurality of individuals and the trait information on the plurality of individuals; and

a predicting step for predicting trait information on an individual from genetic information on the individual based on the relationship between the genetic information and the trait information.

### [Item A2-1]

A method for predicting trait information on an individual, comprising:

an information providing step for providing genetic information on a plurality of individuals and trait information on the plurality of individuals, the genetic information containing at least two types of information;

a learning step for learning a relationship between genetic information and trait information from the genetic information on the plurality of individuals and the trait information on the plurality of individuals;

a predicting step for predicting trait information on an individual from genetic information on the individual based on the relationship between the genetic information and the trait information; and

a displaying step for displaying the predicted trait information.

### [Item A3-1]

The method of any of the preceding items, further comprising a feature of any one or more of the preceding items.

### [Item A1-2]

A program causing a computer to execute a method for predicting trait information on an individual, the method comprising:

an information providing step for providing genetic information on a plurality of individuals and trait information on the plurality of individuals, the genetic information containing at least two types of information;

a learning step for learning a relationship between genetic information and trait information from the genetic information on the plurality of individuals and the trait information on the plurality of individuals; and

a predicting step for predicting trait information on an individual from genetic information on the individual based on the relationship between the genetic information and the trait information.

### [Item A2-2]

The program of the preceding item, the method further comprising a displaying step for displaying the predicted trait information.

### [Item A3-2]

The program of any of the preceding items, further comprising a feature of any one or more of the preceding items.

### [Item A1-3]

A recording medium storing a program causing a computer to execute a method for predicting trait information on an individual, the method comprising:

an information providing step for providing genetic information on a plurality of individuals and trait information on the plurality of individuals, the genetic information containing at least two types of information;

a learning step for learning a relationship between genetic information and trait information from the genetic information on the plurality of individuals and the trait information on the plurality of individuals; and

a predicting step for predicting trait information on an individual from genetic information on the individual based on the relationship between the genetic information and the trait information.

### [Item A2-3]

The recording medium of any of the preceding items, the method further comprising a displaying step for displaying the predicted trait information.

### [Item A3-3]

The recording medium of any of the preceding items, further comprising a feature of any one or more of the preceding items.

### [Item B1]

A method of forming an image of sequence data for a genetic factor population comprising a plurality of genetic factors and expression data for a genetic factor population comprising a plurality of genetic factors, comprising the step of:

generating image data for storing the sequence data for the genetic factor population and the expression data for the genetic factor population, the image data having a plurality of pixels, each of which comprising position information and color information.

### [Item B2]

The method of the preceding item, wherein each of the plurality of genetic factors is associated with a region in the image data, the step of generating the image data comprising the step of:

converting an amount of expression of the genetic factor into color information in a certain region within a region associated with the genetic factor and/or information on an area of a region having a certain color in the region.

### [Item B2-1]

A program causing a computer to execute a method of forming an image of sequence data for a genetic factor population comprising a plurality of genetic factors and expression data for a genetic factor population comprising a plurality of genetic factors, the method comprising the step of:

generating image data for storing the sequence data for the genetic factor population and the expression data for the genetic factor population, the image data having a plurality of pixels, each of which comprising position information and color information.

### [Item B3]

A method of forming an image of genetic information, the genetic information containing sequence data and/or expression data for a genetic factor population comprising a plurality of genetic factors, the method comprising the step of:

generating image data for storing the sequence data and/or expression data for the genetic factor population, the image data having a plurality of pixels, each of which comprising position information and color information,

wherein the step comprises associating each of the plurality of genetic factors with a region in the image data, and regions associated with each genetic factor are arranged so that those with a high correlation weighting of each genetic factor are in proximity.

### [Item B4]

The method of the preceding item, wherein the step of generating the image data further comprises computing an area of a region in image data that is required for the genetic factor.

### [Item B4-1]

A program causing a computer to execute a method of forming an image of genetic information, the genetic information containing sequence data and/or expression data for a genetic factor population comprising a plurality of genetic factors, the method comprising the step of:

generating image data for storing the sequence data and/or expression data for the genetic factor population, the image data having a plurality of pixels, each of which comprising position information and color information,

wherein the step comprises associating each of the plurality of genetic factors with a region in the image data, and regions associated with each genetic factor are arranged so that those with a high correlation weighting of each genetic factor are in proximity.

### [Item B5]

The method of any of the preceding items, wherein the correlation weighting is computed by:

extracting a combination of genetic factors with a strong correlation from correlation analysis between genetic factors;

extracting a genetic factor with a strong correlation for each of the genetic factors;

performing variable selection multiple regression using the extracted genetic factors, and

computing a correlation weighting from a result of the variable selection multiple regression.

### [Item B6]

The method of any of the preceding items, wherein the sequence data for the genetic factor population comprises a sequence data for a factor associated with an event that propagates a genetic trait from a parent cell to a daughter cell.

### [Item B7]

The method of any of the preceding items, wherein the expression data for the genetic factor population comprises expression data for a factor associated with communication of information for only the current generation.

### [Item B8]

The method of any of the preceding items, wherein the sequence data and expression data are for a genetic factor of the same individual.

### [Item B9]

The method of any of the preceding items, wherein each of the plurality of genetic factors is associated with a region in the image data, and the step of generating the image data comprises the step of:

converting information on a position and a type of a mutation in a sequence of a genetic factor into position and color information within a region associated with the genetic factor.

### [Item B10]

The method of any of the preceding items, wherein the step of generating the image data further comprises the step of:

converting information on a modification in a sequence of a genetic factor into position and color information within a region associated with the genetic factor.

### [Item B11]

The method of any of the preceding items, wherein the expression data for the genetic factor population comprises expression data for a transcription unit.

### [Item B12]

The method of any of the preceding items, wherein the expression data for the genetic factor population comprises expression data for an mRNA.

### [Item B13]

The method of any of the preceding items, wherein the expression data for an mRNA comprises data for an amount of expression, splicing, a transcription start point, and/or an epigenetic modification of the mRNA.

### [Item B14]

The method of any of the preceding items, wherein the expression data for the genetic factor population comprises expression data for an miRNA, an snoRNA, an siRNA, a tRNA, an rRNA, an mitRNA, and/or a long chain non-coding RNA.

### [Item B15]

The method of any of the preceding items, wherein the expression data for the genetic factor population comprises data for an amount of expression, splicing, a transcription start point, and/or an epigenetic modification of an miRNA, an snoRNA, an siRNA, a tRNA, an rRNA, an mitRNA, and/or a long chain non-coding RNA.

### [Item B16]

A method for creating a model for predicting trait information on an individual from sequence information and expression information on a genetic factor of an individual, comprising the steps of:

forming an image of sequence information and expression information on a genetic factor of a plurality of individuals by the method of any one of the preceding items to provide image data;

providing trait information on the plurality of individuals; and

extracting an expression of a feature in an image correlated with a trait from the image data and the trait information by deep learning.

### [Item B1-1]

A program causing a computer to execute a method of forming an image of sequence data for a genetic factor population comprising a plurality of genetic factors and expression data for a genetic factor population comprising a plurality of genetic factors, the method comprising the step of:

generating image data for storing the sequence data for the genetic factor population and the expression data for the genetic factor population, the image data having a plurality of pixels, each of which comprising position information and color information.

### [Item B1-2]

A recording medium storing a program causing a computer to execute a method of forming an image of sequence data for a genetic factor population comprising a plurality of genetic factors and expression data for a genetic factor population comprising a plurality of genetic factors, the method comprising the step of:

generating image data for storing the sequence data for the genetic factor population and the expression data for the genetic factor population, the image data having a plurality of pixels, each of which comprising position information and color information.

### [Item B1-3]

A system for executing a method of forming an image of sequence data for a genetic factor population comprising a plurality of genetic factors and expression data for a genetic factor population comprising a plurality of genetic factors, the system comprising:

an image generation unit for generating image data for storing the sequence data for the genetic factor population and the expression data for the genetic factor population, the image data having a plurality of pixels, each of which comprising position information and color information; and

a data storage unit for storing the sequence data for the genetic factor population, the expression data for the genetic factor population, and the image data.

### [Item B16-1]

A program causing a computer to execute a method of creating a model for predicting trait information on an individual from sequence information and expression information on a genetic factor of an individual, the method comprising the steps of:

forming an image of sequence information and expression information on a genetic factor of a plurality of individuals by the method of any one of items B1 to B15 to provide image data;

providing trait information on the plurality of individuals; and

extracting an expression of a feature in an image correlated with a trait from the image data and the trait information by deep learning.

### [Item B16-2]

A recording medium storing a program causing a computer to execute a method of creating a model for predicting trait information on an individual from sequence information and expression information on a genetic factor of the individual, the method comprising the steps of:

forming an image of sequence information and expression information on a genetic factor of a plurality of individuals by the method of any one of the preceding items to provide image data;

providing trait information on the plurality of individuals; and

extracting an expression of a feature in an image correlated with a trait from the image data and the trait information by deep learning.

### [Item B16-3]

A system for executing a method for creating a model for predicting trait information on an individual from sequence information and expression information on a genetic factor of the individual, the system comprising:

an image generation unit for forming an image of sequence information and expression information on a genetic factor of a plurality of individuals by the method of any one of the preceding items to provide image data;

a data storage unit for storing trait information on the plurality of individuals and the image data; and

a learning unit for extracting an expression of a feature in an image that is correlated with a trait from the image data and the trait information by deep learning.

### [Item C1]

A data structure of image data representing sequence information on a genetic factor population comprising a plurality of genetic factors and expression information on a genetic factor population comprising a plurality of genetic factors, wherein

the image data has a plurality of regions associated with the plurality of genetic factors;

each position in a sequence of a genetic factor is associated with a position within the regions associated with the genetic factor;

information on a substitution, a deletion, and/or an insertion at each position in the sequence of the genetic factor is stored as color information at a position associated with the position; and

expression data for the genetic factor is stored as color information at a certain region in the regions, and/or information on an area of a region having a certain color in the regions.

### [Item C2]

The data structure of the preceding item, wherein

information on an epigenetic modification at each position in a sequence of the genetic factors is further stored as color information at a position associated with the position.

### [Item C3]

The data structure of any of the preceding items, wherein methylation at each position in a sequence of an miRNA in the plurality of genetic factors is stored as color information at a position associated with the position.

### [Item C4]

The data structure of any of the preceding items, wherein the image data is a matrix having a row and a column, and each of the positions is stored as a combination of a row and a column.

### [Item C5]

A data structure of image data representing sequence information and expression information, the image data being a matrix having a row and a column, and each position in the image data being stored as a combination of a row and a column, wherein

the sequence information contains a DNA sequence of a region on a genome, and the region on the genome comprises a gene, an exon, an intron, a non-expression region, and/or a non-coding RNA encoding region;

the expression information comprises information on an amount of expression, splicing, a transcription start point, and/or an epigenetic modification of a transcription unit selected from the group consisting of an mRNA, an miRNA, an snoRNA, an siRNA, a tRNA, an rRNA, an mitRNA, and/or a long chain non-coding RNA;

the image data has a plurality of regions associated with a region and/or transcription unit on each genome;

the regions associated with a region on the genome consist of a number of columns dependent on a length of the region on the genome and a certain number of rows;

each position in a sequence of the region on the genome is associated with a position in an odd number column within the regions associated with a region on the genome;

information on a substitution, a deletion, and/or an insertion at each position in the sequence of a region on the genome is stored as color information at a position in an odd number column associated with the position, and the color information is color information indicating the absence of a mutation, color information indicating a substitution with A, color information indicating a substitution with T, color information indicating a substitution with G, color information indicating a substitution with C, color information indicating the presence of a deletion, or color information indicating the presence of an insertion adjacent to the position;

color information indicating an inserted sequence is stored as information on the inserted sequence, with a position in an even number column adjacent to a position having color information indicating the presence of an insertion as a starting point;

information on an epigenetic modification at each position in a sequence of a region on the genome is stored as color information at a position in an odd number column associated with the position, and the color information comprises color information indicating the absence of an epigenetic modification, color information indicating DNA methylation, color information indicating histone methylation, color information indicating histone acetylation, color information indicating histone ubiquitination, or color information indicating histone phosphorylation;

an amount of expression of a transcription unit transcribed from a region on a genome is stored as a shade of a color in a region in an image associated with a region on the genome and/or information on an area of a region having a certain color in the region; and

an amount of expression of an mRNA associated with a gene for a region on a genome that is the gene is stored as a shade of a color in a region in the region and/or information on an area of a region having a certain color in the region.

### [Item D1]

A method for creating a model for predicting a relationship between an image and information associated with the image, comprising the steps of:

providing a set of a plurality of images and a plurality of pieces of information associated with the plurality of images;

obtaining a plurality of divided learning data by dividing the plurality of images and learning a relationship between a portion of the plurality of images and information associated with the images; and

integrating the plurality of divided learning data to generate a model for predicting the relationship between the image and the information associated with the image.

### [Item D2]

The method of the preceding item, wherein the integration step comprises detecting a GPU specification and a CPU specification comprising an amount of on-board memory using a CPU machine with a GPU installed therein.

### [Item D3]

The method of any of the preceding items, wherein the integration step comprises optimizing a non-linear optimization processing algorithm that can utilize a Read-Write file on an HDD and utilize a CPU memory as much as possible.

### [Item D4]

The method of any of the preceding items, wherein the non-linear optimization processing algorithm is an algorithm capable of calculation independent of data size by transferring required data to a memory as needed to perform a calculation, and returning a calculation result to an HDD.

### [Item D5]

The method of any of the preceding items, wherein the non-linear optimization processing comprises optimizing a full differentiation parameter.

### [Item D6]

The method of any of the preceding items, wherein the step of obtaining a plurality of divided learning data verifies an ability to differentiate each divided learning data, selects divided learning data with an ability to differentiate, and subjects the data to integration.

### [Item D1-1]

A program causing a computer to execute a method for creating a model for predicting a relationship between an image and information associated with the image, the method comprising the steps of:

providing a set of a plurality of images and a plurality of pieces of information associated with the plurality of images;

obtaining a plurality of divided learning data by dividing the plurality of images and learning a relationship between a portion of the plurality of images and information associated with the images; and

integrating the plurality of divided learning data to generate a model for predicting the relationship between the image and the information associated with the image.

### [Item D1-2]

A recording medium storing a program causing a computer to execute a method for creating a model for predicting a relationship between an image and information associated with the image, the method comprising the steps of:

providing a set of a plurality of images and a plurality of pieces of information associated with the plurality of images;

obtaining a plurality of divided learning data by dividing the plurality of images and learning a relationship between a portion of the plurality of images and information associated with the images; and

integrating the plurality of divided learning data to generate a model for predicting the relationship between the image and the information associated with the image.

### [Item D1-2]

A system for creating a model for predicting a relationship between an image and information associated with the image, the system comprising:

a data storage unit for providing a set of a plurality of images and a plurality of pieces of information associated with the plurality of images;

a data learning unit for obtaining a plurality of divided learning data by dividing the plurality of images and learning a relationship between a portion of the plurality of images and information associated with the images; and

a model generation unit for integrating the plurality of divided learning data to generate a model for predicting the relationship between the image and the information associated with the image.

### [Item E1]

A system for predicting trait information on an individual, comprising:

a storage unit for storing genetic information on a plurality of individuals and trait information on the plurality of individuals, the genetic information containing sequence information and expression information on a genetic factor;

a learning unit configured to learn a relationship between genetic information and trait information from the genetic information on the plurality of individuals and the trait information on the plurality of individuals by forming an image of the genetic information on the plurality of individuals; and

a calculation unit for predicting trait information on an individual from genetic information on the individual based on the relationship between the genetic information and the trait information;

wherein the learning unit is configured to divide an image generated by forming an image of the genetic information on the plurality of individuals, learn a relationship between each region of the image and trait information, select a region wherein a model with an ability to differentiate trait information can be generated from each region, and generate a model for predicting trait information from each region on the image.

### [Item E2]

A method for creating a model for predicting a relationship between genetic information containing sequence information and expression information on a genetic factor of an individual and trait information on the individual, comprising the steps of:

providing a set of a plurality of images formed from sequence information and expression information on a genetic factor of a plurality of individuals and a plurality of pieces of trait information associated with the plurality of images;

obtaining a plurality of divided learning data by dividing the plurality of images and learning a relationship between a portion of the plurality of images and the information associated with the images; and

selecting divided learning data with an ability to differentiate trait information from the plurality of divided learning data to generate a model for predicting trait information from each region of the images.

### [Item E3]

A program causing a computer to execute a method for creating a model for predicting a relationship between genetic information containing sequence information and expression information on a genetic factor of an individual and trait information on the individual, the method comprising the steps of:

providing a set of a plurality of images formed from sequence information and expression information on a genetic factor of a plurality of individuals and a plurality of pieces of trait information associated with the plurality of images;

obtaining a plurality of divided learning data by dividing the plurality of images and learning a relationship between a portion of the plurality of images and information associated with the images; and

selecting divided learning data with an ability to differentiate trait information from the plurality of divided learning data to generate a model for predicting trait information from each region of the images.

### [Item F1]

A system for predicting trait information on an individual, comprising:

a storage unit for storing genetic information on a plurality of individuals and trait information on the plurality of individuals, the genetic information containing sequence information and expression information on a genetic factor;

a learning unit configured to learn a relationship between genetic information and trait information from the genetic information on the plurality of individuals and the trait information on the plurality of individuals by forming an image of the genetic information on the plurality of individuals; and

a calculation unit for predicting trait information on an individual from genetic information on the individual based on the relationship between the genetic information and the trait information;

wherein the learning unit is configured to divide an image generated by forming an image of the genetic information on the plurality of individuals, learn a relationship between each region of the image and trait information, select a region where a model with an ability to differentiate trait information can be generated from each region, determine whether trait information can be predicted based on expression information in each region, and identify a gene having a mutation that is correlated with trait information from a gene in a region where trait information cannot be predicted based on expression information, and the calculation unit is configured to predict the trait information on the individual based on information on the gene having a mutation that is correlated with the trait information.

### [Item F1-1]

The system of the preceding item, wherein the determination of whether trait information can be predicted based on expression information is performed by:

performing cluster analysis on the plurality of individuals based on each amount of expression of a gene contained in each region of the image;

dividing the plurality of individuals into groups in accordance with trait information;

computing identity between the groups and clusters divided by cluster analysis; and

determining that trait information can be predicted based on expression information when the identity exceeds a given threshold value (e.g., 80 to 90%).

### [Item F1-2]

The system of any of the preceding items, wherein the learning unit is configured to further divide a region where trait information can be predicted based on expression information after determining whether trait information can be predicted based on expression information and further determine whether trait information can be predicted based on expression information for each divided region, and is configured to identify a gene having a mutation that is correlated with trait information from a region where it is possible to differentiate from only information on an amount of gene expression.

### [Item F1-3]

The system of any of the preceding items, wherein the identification of a gene having a mutation that is correlated with trait information from a gene in a region where trait information cannot be predicted based on expression information further comprises further dividing the region and narrowing down a region where trait information cannot be predicted based on expression information.

### [Item F2]

A method for identifying a mutation of a gene associated with a trait, comprising the steps of:

providing a set of a plurality of images formed from sequence information and expression information on a genetic factor of a plurality of individuals and a plurality of pieces of trait information associated with the plurality of images;

obtaining a plurality of divided learning data by dividing the plurality of images and learning a relationship between a portion of the plurality of images and information associated with the images; and

selecting a portion of an image where divided learning data with an ability to differentiate trait information can be obtained;

determining whether trait information can be predicted based on expression information from the portion of an image where divided learning data with an ability to differentiate trait information can be obtained to select a portion where trait information cannot be predicted based on expression information; and

identifying a gene having a mutation that is correlated with trait information from a gene contained at the portion where trait information cannot be predicted based on expression information.

### [Item F2-1]

The method of the preceding item, wherein the determining whether trait information can be predicted based on expression is performed by:

performing cluster analysis on the plurality of individuals based on each amount of expression of a gene contained in each region of the image;

dividing the plurality of individuals into groups in accordance with trait information;

computing identity between the groups and clusters divided by cluster analysis; and

determining that trait information can be predicted based on expression information when the identity exceeds a given threshold value (e.g., 80 to 90%).

### [Item F2-2]

The method of any of the preceding items, further comprising further dividing a region where trait information can be predicted based on expression information after determining whether trait information can be predicted based on expression information, further determining whether trait information can be predicted based on expression information for each divided region, and identifying a gene having a mutation that is correlated from trait information from a region where it is possible to differentiate from only information an amount of gene expression.

### [Item F2-3]

The method of any of the preceding items, wherein the identification of a gene having a mutation that is correlated with trait information from a gene in a region where trait information cannot be predicted based on expression information further comprises further dividing the region and narrowing down a region where trait information cannot be predicted based on expression information.

### [Item F3]

A program causing a computer to execute a method for identifying a mutation of a gene associated with a trait, the method comprising the steps of:

providing a set of a plurality of images formed from sequence information and expression information on a genetic factor of a plurality of individuals and a plurality of pieces of trait information associated with the plurality of images;

obtaining a plurality of divided learning data by dividing the plurality of images and learning a relationship between a portion of the plurality of images and information associated with the images;

selecting a portion of an image where divided learning data with an ability to differentiate trait information can be obtained;

determining whether trait information can be predicted based on expression information from the portion of an image where divided learning data with an ability to differentiate trait information can be obtained to select a portion where trait information cannot be predicted based on expression information; and

identifying a gene having a mutation that is correlated with trait information from a gene contained at the portion where trait information cannot be predicted based on expression information.

### [Item F3-1]

The program of the preceding item, wherein the determination of whether trait information can be predicted based on expression information is performed by:

performing cluster analysis on the plurality of individuals based on each amount of expression of a gene contained in each region of the image;

dividing the plurality of individuals into groups in accordance with trait information;

computing identity between the groups and clusters divided by cluster analysis; and

determining that trait information can be predicted based on expression information when the identity exceeds a given threshold value (e.g., 80 to 90%).

### [Item F3-2]

The program of any of the preceding items, further comprising further dividing a region where trait information can be predicted based on expression information after determining whether trait information can be predicted based on expression information, further determining whether trait information can be predicted based on expression information for each divided region, and identifying a gene having a mutation that is correlated from trait information from a region where it is possible to differentiate from only information an amount of gene expression.

### [Item F3-3]

The program of any of the preceding items, wherein the identification of a gene having a mutation that is correlated with trait information from a gene in a region where trait information cannot be predicted based on expression information further comprises further dividing the region and narrowing down a region where trait information cannot be predicted based on expression information.

### Advantageous Effects of Invention

The present disclosure provides means for predicting trait information on an individual from data for genetic information on the individual. The means is useful in any technical field related to organisms such as the medical, agricultural, animal husbandry, food, environmental, and pharmaceutical (drug development and postmarketing surveillance) fields. This enables information on the possibility of developing a disease, suitable therapy, expected response, or the like to be provided, especially in the medical field. In addition, the machine learning method according to the present disclosure can enable the handling of an enormous amount of data in any machine learning using an image.

### BRIEF DESCRIPTION OF DRAWINGS

FIG. 1 is an exemplary schematic diagram of the system of the present disclosure.

FIG. 2 is a diagram of the system of the present disclosure which is physically separated by using a cloud/server, etc.

FIG. 3 is an exemplary schematic diagram of a step of performing machine learning on DNA/RNA data.

FIG. 4 is an exemplary schematic diagram of a step of forming an image of DNA/RNA data.

FIG. 5 is an exemplary schematic diagram of optimization of arrangement when forming an image of DNA/RNA data.

FIG. 6 is an exemplary schematic diagram of correlation analysis between genes for optimization of arrangement.

FIG. 7 is an exemplary schematic diagram of Deep Learning processing in learning a divided image.

FIG. 8 is an exemplary schematic diagram of GPU divided learning and CPU non-linear optimization.

FIG. 9 is a graph showing the percentage of correct answers at each number of epochs of a generated model. The constructed differentiation model was able to differentiate at a 100% accuracy on cell lines using a non-learned image.

FIG. 10 is a graph showing the differentiability with an image used upon learning and the differentiability with an image that was not used upon learning at each number of epochs for each of the models generated by machine learning each of an image formed from both DNA mutation data and RNA expression level data, an image formed in the same manner from information on only DNA mutation data, and an image formed in the same manner from information on only RNA expression level data.

FIG. 11 is a schematic diagram showing learning from dividing an image.

FIG. 12 is a diagram showing the difference in region convergence upon learning 5FU sensitivity.

### DESCRIPTION OF EMBODIMENTS

The present disclosure is described hereinafter while showing the best mode of the disclosure. Throughout the entire specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form, unless specifically noted otherwise. The terms used herein should also be understood as being used in the meaning that is commonly used in the art, unless specifically noted otherwise. Thus, unless defined otherwise, all terminologies and scientific technical terms that are used herein have the same meaning as the general understanding of those skilled in the art to which the present invention pertains. In case of a contradiction, the present specification (including the definitions) takes precedence.

The definitions of the terms and/or the detailed basic technology that are particularly used herein are described hereinafter as appropriate.

### Definitions

As used herein, “full differentiation parameter” refers to a parameter in a differentiation formula for differentiating an entire image integrated after divided learning. A differentiation analysis formula in individual learning differentiates by adding weighting to partial data for a divided image. Thus, completely independent differentiation formulas are used for each divided image, so that there is no correlation therebetween. Therefore, the final non-linear optimization creates a new differentiation formula (for the entire image prior to dividing) that integrates differentiation formulas using a parameter found in each partial learning. For this reason, a process of optimizing the whole using a CPU is performed, with a parameter from each partial learning as an initial value.

As used herein, “on the fly” processing refers to processing that repeatedly transfers required data to a memory as needed to perform calculation, and returns a calculation result to an HDD. “On the fly” can be understood by comparing a memory to a bookshelf next to a desk and HDD to a library. When processing at a desk, a book, which is data, can be processed quickly if the book is in the adjacent bookshelf. Generally, all the books that are needed are brought to the bookshelf together. However, the bookshelf size is limited, so that required data (book) can be transferred to a memory (bookshelf) as needed to perform a calculation and returned to an HDD (library), and repeatedly transfer, calculate, and return to handle a large volume of books. Examples employing “on the fly” processing in the optimization processing in the present disclosure include a case employing an algorithm that is not time efficient in memory communication, but is capable of calculating any sized learning data (even with a compromise in calculation time) during the optimization processing.

As used herein, “image” refers to, as broadly defined, any data stored in a high-dimensional space, and particularly, as narrowly defined, data stored on a plane (two-dimensional space). Examples of narrowly defined images include a combination of position information and color (hue, brightness, or saturation) information at each position. “Image formation” refers to converting one dimensionally stored data (e.g., column of 0 and 1) into data stored in a higher dimension.

As used herein, “learning” refers to forming a model that provides a useful output in response to an input using some type of data. When an input and a corresponding output are used as learning data, this is referred to as “supervised learning”. Examples of models include a model that outputs a trait (e.g., drug resistance) estimated from genetic information when the genetic information is used as an input, and the like.

As used herein, “trait information” refers to information on any feature of an organism or a part of an organism (e.g., organ, tissue, or cell). Examples of trait information include specifics of diseases (e.g., for cancer, specific cancer type, grade or malignancy of cancer, etc.), drug sensitivity (e.g., for cancer, anticancer agent resistance), and the like.

As used herein, “genetic factor” refers to any factor that carries out some type of function based on information during the activity of an organism. For example, a gene on a genomic DNA is a genetic factor in terms of being transcribed into a corresponding mRNA based on the information on the sequence thereof. An mRNA is also a genetic factor in terms of being translated into a corresponding protein or the like based on the information on the sequence thereof. Genetic factors comprehensively encompass factors encoding miRNA, regulatory region, non-expression region, and the like in addition to genes encoding a protein. Therefore, as used herein, “genetic factor” encompasses exons, introns, non-expression regions, non-coding RNAs, miRNAs, snoRNAs, siRNAs, tRNAs, rRNAs, mitRNAs, and long chain non-coding RNAs in addition to genes and mRNAs.

As used herein, “genetic information” refers to sequence information and/or expression information on any genetic factor of an organism or a part of an organism (e.g., tissue or cell).

As used herein, “ribonucleic acid (RNA)” refers to a molecule comprising at least one ribonuleotide residue. “Ribonucleotide” refers to a nucleotide having a hydroxyl group at position 2′ in the β-D-ribofuranose moiety. Examples of RNAs include messenger RNA (mRNA), transfer RNA (tRNA), ribosomal RNA (rRNA), long non-coding RNA (lncRNA), and microRNA (miRNA).

As used herein, “deoxyribonucleic acid (DNA)” refers to a molecule comprising at least one deoxyribonucleotide residue. “Deoxyribonucleotide” refers to a nucleotide with a hydroxyl group at position 2′ of a ribonucleotide substituted with hydrogen.

As used herein, “messenger RNA (mRNA)” refers to an RNA prepared by using a DNA template and is associated with a transcript encoding a peptide or polypeptide. Typically, an mRNA comprises 5′-UTR, protein coding region, and 3′-UTR. Specific information (sequence and the like) on mRNAs is available from, for example, NCBI (https://www.ncbi.nlm.nih.gov/).

As used herein, “microRNA (miRNA)” refers to a functional nucleic acid, which is encoded on the genome and ultimately becomes a very small RNA with a base length of 20 to 25 after undergoing a multi-stage production process. Specific information (sequence and the like) on miRNAs is available from, for example, mirbase (http://mirbase.org).

As used herein, “long non-coding RNA (lncRNA)” refers to an RNA of 200 nt or greater that functions without being translated into a protein. Specific information (sequence and the like) on lncRNAs is available from, for example, RNAcentral (http://rnacentral.org/).

As used herein, “ribosomal RNA (rRNA)” refers to an RNA constituting a ribosome. Specific information (sequence and the like) on rRNAs is available from, for example, NCBI (https://www.ncbi.nlm.nih.gov/).

As used herein, “transfer RNA (tRNA)” refers to a tRNA that is known to be aminoacylated by an aminoacyl tRNA synthetase. Specific information (sequence and the like) on tRNAs is available from, for example, NCBI (https://www.ncbi.nlm.nih.gov/).

As used herein, “modification” used in the context of a nucleic acid refers to a substitution of a constituent unit of a nucleic acid or a part or all of the terminus thereof with another group of atoms, or addition of a functional group. A collection of modifications of an RNA is also known as “RNA Modomics”, “RNA Mod”, or the like, which are also known as epitranscriptome because an RNA is a transcript. These terms are used synonymously herein.

As used herein, “methylation” used in the context of a nucleic acid refers to methylation of any location of any type of nucleotide and is typically methylation of adenine (e.g., position 6; m6A, position 1; m1A) or methylation of cytosine (e.g., position 5; m5C, position 3; m3C). A detected modified site can be identified using a methodology that is known in the art. For example, each of m1A and m6A and m3C and m5C can be determined by chemical modifications. For example, it is possible to determine whether a behavior according to measurement by MALDI and chemical modification is correct by utilizing a standard synthetic RNA.

As used herein, “subject” refers to a subject targeted for the analysis, diagnosis, detection, or the like of the present disclosure (e.g., organism such as a human or cell, blood, or serum retrieved from an organism, or the like).

As used herein, “biomarker” is an indicator for evaluating a condition or action of a subject. Unless specifically noted otherwise, “biomarker” is also referred to as “marker” herein.

As used herein, “diagnosis” refers to identifying various parameters associated with a condition (e.g., disease or disorder) in a subject or the like to determine the current or future state of such a condition. The condition in the body can be investigated by using the method, apparatus, or system of the present disclosure. Such information can be used to select and determine various parameters of a metastatic/primary condition of cancer in a subject (e.g., whether the subject has metastatic cancer, or the cancer is primary cancer), a formulation or method for the treatment or prevention to be administered, or the like. As used herein, “diagnosis” when narrowly defined refers to diagnosis of the current state, but when broadly defined includes “early diagnosis”, “predictive diagnosis”, “prediagnosis”, and the like. Since the diagnostic method of the present disclosure in principle can utilize what comes out from a body and can be conducted away from a medical practitioner such as a physician, the present disclosure is industrially useful. In order to clarify that the method can be conducted away from a medical practitioner such as a physician, the term as used herein may be particularly called “assisting” “predictive diagnosis, prediagnosis, or diagnosis”. The technology of the present disclosure can be applied to such a diagnostic technology.

As used herein, “therapy” refers to the prevention of exacerbation, preferably maintaining of the current condition, more preferably alleviation, and still more preferably disappearance of a condition (e.g., disease or disorder) in case of developing such a condition, including being capable of exerting a prophylactic effect or an effect of improving a condition of a patient or one or more symptoms accompanying the condition. Preliminary diagnosis with suitable therapy is referred to as “companion therapy” and a diagnostic agent therefor may be referred to as “companion diagnostic agent”. Using the technology of the present disclosure to associate genetic information with diagnostically useful trait information can be useful in such companion therapy of companion diagnosis.

As used herein, “prevention” refers to treatment to avoid reaching a non-normal state (e.g., disease or disorder).

The term “prognosis” as used herein refers to prediction of the possibility of death due to a disease, disorder, or the like such as cancer or progression thereof. A prognostic agent is a variable related to the natural course of a disease or disorder, which affects the rate of recurrence or the like in a patient who has developed the disease or disorder. Examples of clinical indicators associated with exacerbation in prognosis include any cell indicator used in the present disclosure. A prognostic agent is often used to classify patients into subgroups with different pathological conditions. Associating genetic information with diagnostically useful trait information using the technology of the present disclosure can enable a prognostic agent to be provided based on genetic information of the control.

As used herein, “program” is used in the meaning that is commonly used in the art. A program describes the processing to be performed by a computer in order, and is legally considered a “product”. All computers operate in accordance with a program. Programs are expressed as data in modern computers and are stored in a recording medium or a storage device.

As used herein, “recording medium” is a medium storing a program for executing the present disclosure. A recording medium can be anything, as long as a program can be recorded. Examples thereof include, but are not limited to, a ROM or HDD or a magnetic disk that can be stored internally, or an external storage device such as flash memory such as a USB memory.

As used herein, “system” refers to a configuration that executes the method of program of the present disclosure. A system fundamentally means a system or organization for executing an objective, wherein a plurality of elements are systematically configured to affect one another. In the field of computers, system refers to the entire configuration such as the hardware, software, OS, and network.

(Prediction System)

One aspect of the present disclosure is a system for predicting trait information on an individual. The system can comprise a storage unit for storing genetic information on a plurality of individuals and trait information on the plurality of individuals; a learning unit configured to learn a relationship between genetic information and trait information from the genetic information on the plurality of individuals and the trait information on the plurality of individuals; and a calculation unit for predicting trait information on an individual from genetic information on the individual based on the relationship between the genetic information and the trait information. In one embodiment, the genetic information contained in the storage unit can contain at least two types of information. Optionally, the system can further comprise an analysis unit for analyzing diagnosis of the individual and/or treatment or prophylaxis on the individual from the trait information predicted in the calculation unit. Optionally, the system can further comprise a display unit for displaying the trait information predicted in the calculation unit.

The present disclosure can also be provided as a program or a method that materializes the system described above or a recording medium storing the same.

The learning unit can be configured to learn after forming an image of the genetic information on the plurality of individuals. At the same time, an image of genetic information on a plurality of individuals can be formed and stored in the storage unit. In another embodiment, an image can be formed each time upon learning. The calculation unit can also form an image of genetic information on an individual and predict trait information on the individual based on the information. An image can be formed by a method or system with the feature described elsewhere herein. Image data may also have a data format described elsewhere herein. The system can comprise other constituent elements as needed. For example, the system can comprise a display unit for displaying an output of the calculation unit.

One embodiment performs learning using artificial intelligence (AI) as the learning. While AI technologies are known to be capable of high performance through extraction of expression of a feature in processing data such as an “image” or audio, the technologies are considered as still having issues with other types of data. One issue is that, as demonstrated in previous cellular biological studies, “morphological” information of a cell is very important, but directly linking such morphological information to genomic information required finding statistical correlation from visual inspection of numerical data and image of the genome through a method such as sequencing, single cell analysis, or the like in conventional methods. However, the present invention “forms an image” of genomic information to provide genomic information in the same form as images to allow comparison between images, so that maximum performance of AI can be expected.

When the subject is a human, it is socially critical that genetic information is in compliance from the viewpoint of personal information. From this viewpoint, formation of an image of genomic information has the potential to be one of the fundamental technologies for “privacy shield”. If image formation includes extracting mutation information and creating a database and is set to allow SNPs in such a case, this can be a shield against identification of an individual. Specifically, it is understood that mutation information alone cannot be a code for identifying an individual.

Examples of genetic information used in the present disclosure include sequence information (e.g., mutation information), expression information, and/or modification information (e.g., methylation information) on a genetic factor. Data from a plurality of individuals is generally required as data used in learning, but it is not necessary to obtain every type of genetic information from each individual.

A factor associated with an event that propagates a genetic trait from a parent cell to a daughter cell in the nucleus or mitochondria under the control of an RNA polymerase, which is a DNA sequence encoding not only a coding RNA or mRNA encoding a protein, but also miRNA, snoRNA, siRNA, tRNA, rRNA, mitRNA with a relatively short strand up to 10s of bases, as well as longer chain non-coding RNA as non-coding RNA, can be targeted as sequence information, as genetic information. A DNA sequence of a non-expression region away from a complimentary portion of the expression product described above as well as epigenetic modification on a DNA or the like can also be targeted. As expression information on an individual, a DNA sequence encoding not only a coding RNA or mRNA encoding a protein, but also miRNA, snoRNA, siRNA, tRNA, rRNA, or mitRNA with a relatively short strand up to 10s of bases as well as longer chain non-coding RNA as non-coding RNA, under the control of an RNA polymerase, including a genetic factor of an individual (an amount of expression, splicing, a transcription start point, an epigenetic modification, and the like of a transcription unit (RNA and miRNA)) can be targeted.

Examples of trait information used in the present disclosure include, but are not particularly limited to, whether an individual can develop a certain disease, whether an individual is responsive to a certain agent, and the like.

The storage unit can be a recording medium that is stored in the system or external to the system, such as CD-R, DVD, Blu-ray, USB, SSD, or hard disk. Alternatively, the storage unit can be stored in a server or configured to be appropriately recorded in the cloud.

The learning unit can be configured to learn the relationship between genetic information and trait information by using artificial intelligence or machine learning. As used herein, “machine learning” refers to a technology for imparting a computer with the ability to learn without explicit programming. This is a process of improving a function unit's own performance by acquiring new knowledge/skill or reconstituting existing knowledge/skill. Most of the effort required for programming details can be reduced by programming a computer to learn from experience. In the machine learning field, a method of constructing a computer program that enables automatic improvement from experience has been discussed. Data analysis/machine learning plays a role as elemental technology that is the foundation of intelligent processing along with a field of the algorithms. Generally, data analysis/machine learning is utilized in conjunction with other technologies, thus requiring the knowledge in the linked field (domain specific knowledge; e.g., medical field). The range of application thereof includes roles such as prediction (collect data and predict what would happen in the future), search (find a notable feature from collected data), and testing/describing (find relationship of various elements in the data). Machine learning is based on an indicator indicating the degree of achievement of a goal in the real world. The user of machine learning must understand the goal in the real world. An indicator that improves when an objective is achieved needs to be formularized. Machine learning has the opposite problem that is an ill-posed problem for which it is unclear whether a solution is found. The behavior of the learned rule is not definitive, but is stochastic (probabilistic). Machine learning requires an innovative operation with the premise that some type of uncontrollable element would remain. It is useful for a user of machine learning to successively pick and choose data or information in accordance with the real world goal while observing performance indicators during training and operation.

Linear regression, logistic regression, support vector machine, or the like can be used for machine learning, and cross validation (CV) can be performed to compute differentiation accuracy of each model. After ranking, a feature can be increased one at a time for machine learning (linear regression, logistic regression, support vector machine, or the like) and cross validation to compute the differentiation accuracy of each model. A model with the highest accuracy can be selected thereby. Any machine learning can be used herein. Linear, logistic, support vector machine (SVM), or the like can be used as supervised machine learning.

Machine learning uses logical reasoning. There are roughly three types of logical reasoning, i.e., deduction, induction, abduction, and analogy. Deduction, under the hypothesis that Socrates is human and all humans die, reaches a conclusion that Socrates would die, which is a special conclusion. Induction, under the hypothesis that Socrates would die and Socrates is human, reaches a conclusion that all humans would die, and derives a general rule. Abduction, under a hypothesis that Socrates would die and all humans die, arrives at Socrates is human, which falls under a hypothesis/explanation. However, it should be noted that how induction generalizes is dependent on the premise, so that this may not be objective. Analogy is a probabilistic logical reasoning method which reasons that, for subject A and subject B, if subject A has four features and subject B has three of the same features, subject B also has the remaining one feature so that subject A and subject B are the same or similar and close.

Impossible has three basic principles, i.e., impossible, very difficult, and unsolved. Further, impossible includes generalization error, no free lunch theorem, and ugly duckling theorem, and true model observation is impossible, so that this is impossible to verify. Such an ill-posed problem should be noted.

Feature/attribute in machine learning represents the state of a subject being predicted when viewed from a certain aspect. A feature vector/attribute vector combines features (attributes) describing a subject being predicted in a vector form.

As used herein, “model” and “hypothesis” are used synonymously, which are expressed using mapping describing the relationship of inputted prediction targets to prediction results, or a mathematical function or Boolean expression of a candidate set thereof. For learning by machine learning, a model considered the best approximation of the true model is selected from a model set by referring to training data.

Examples of models include generation models, identification models, function models, and the like. The models indicate a difference in the direction of expressing a classification model of the mapping relationship between the input (subject being predicted) x and output (result of prediction) y. A generation model expresses a conditional distribution of output y given input x. An identification model expresses a joint distribution of input x and output y. The mapping relationship is probabilistic for an identification model and a generation model. A function model has a definitive mapping relationship, expressing a definitive functional relationship between input x and output y. While identification is sometimes considered slightly more accurate in an identification model and a generation model, there is basically no difference in view of the no free lunch theorem.

Model complexity: the degree of whether mapping relationship of a subject being predicted and a prediction result can be described in more detail and with more complexity. Generally, more training data is required for a model set that is more complex.

If a mapping relationship is expressed as a polynomial equation, a higher order polynomial equation can express a more complex mapping relationship. A higher order polynomial equation is considered a more complex model than a linear equation.

If a mapping relationship is expressed by a decision tree, a deeper decision tree with more nodes can express a more complex mapping relationship. Therefore, a decision tree with more nodes can be considered a more complex model than a decision tree with less nodes.

Classification thereof is also possible by the principle of expressing the corresponding relationship between inputs and outputs. For a parametric model, the shape of the function or distribution is completely determined by parameters. For a nonparametric model, the shape thereof is basically determined from data, and parameters only determine smoothness.

Parameter: an input for designating one of a set of functions or distribution of a model. It is also denoted as Pr[y|x; θ], y=f(x; θ), or the like to distinguish from other inputs.

For a parametric model, the shape of a Gaussian distribution is determined by mean/variance parameters, regardless of the number of training data. For a nonparametric model, only the smoothness is determined by the number of bin parameter in a histogram. This is considered more complex than a parametric model.

For learning by machine learning, a model considered the best approximation of the true model is selected from a model set by referring to training data. There are various learning methods depending on the “approximation” performed. A typical method is the maximum likelihood estimation, which is the standard learning that selects a model with the highest probability of producing training data from a probabilistic model set. Maximum likelihood estimation can select a model that best approximates the true model. KL divergence to the true distribution becomes small for greater likelihood. There are various types of estimation that vary by the type of format for finding a parameter or prediction value that is estimated. Point estimation finds only one value with the highest certainty. Maximum likelihood estimation, MAP estimation, and the like use the mode of a distribution or function and are most often used. Meanwhile, interval estimation is often used in the field of statistics in a form of finding a range within which an estimated value falls, where the probability of an estimated value falling within the range is 95%. Distribution estimation is used in Bayesian estimation or the like in combination with a generation model introduced with a prior distribution for finding a distribution within which an estimated value falls.

In machine learning, over-training (over-fitting) can occur. With over-training, empirical error (prediction error relative to training data) is small, but generalization error (prediction error relative to data from a true model) is large due to selecting a model that is overfitted to training data, such that the original objective of learning cannot be achieved. Generalization errors can be divided into three components, i.e., bias (error resulting from a candidate model set not including a true model; this error is greater for a more simple model set), variance (error resulting from selecting a different prediction model when training data is different; this error is greater for a more complex model set), and noise (deviation of a true model that cannot be fundamentally reduced, which is independent of the choice of a model set). Since bias and variance cannot be simultaneously reduced, the overall error is reduced by balancing the bias and variance.

As used herein, “ensemble (also known as ensemble learning, ensemble method, or the like)” is also referred to as group learning and attempts to perform the same learning as learning of a complex learning model by using a relatively simple learning model and a learning rule with a suitable amount of calculation, and selecting and combining various hypotheses depending on the difference in the initial value or weighting of a given example to construct a final hypothesis. Learning in the present disclosure can be performed by ensemble learning.

As used herein, “contract” refers to reducing or consolidating variables, i.e., features. For example, factor analysis refers to explaining, when there are a plurality of variables, the relationship between a plurality of variables with a small number of potential variables by assuming that there is a constituent concept affecting the variables in the background thereof. This is a form of conversion to a small number of variables, i.e., contracting. The potential variables explaining the constituent concept are referred to as factors. Factor analysis contracts variables that can be presumed to have the same factors in the background to create a new quantitative variable.

As used herein, “differentiation function” is a numerical sequence, i.e., a function, created to match the arrangement of samples to be differentiated by assigning continuous numerical values to the number of levels to be differentiated. For example, if samples to be differentiated are arranged to match the levels when there are two differentiation levels, the numerical sequence thereof, i.e., differentiation function, is generated, for example, to have a form of a sigmoid function. For three or more levels, a step function can be used. A model approximation index numerically represents the relationship between a differentiation function and differentiation level of samples to be differentiated. When a difference therebetween is used, the range of fluctuation is controlled. A smaller absolute value of a differential value indicates higher approximation. When correlation analysis is performed, a higher correlation coefficient (r) indicates higher approximation. When regression analysis is used, a higher R² value is deemed to have higher approximation.

As used herein, “weighting coefficient” is a coefficient that is set so that an important element is calculated as more important in the calculation of the present disclosure, including approximation coefficients. For example, a coefficient can be obtained by approximating a function to data, but the coefficient itself only has a description indicating the degree of approximation. When coefficients are ranked or chosen/discarded on the basis of the magnitude or the like, a difference in contribution within the model is provided to a specific feature, so that this can be considered a weighting coefficient. A weighting coefficient is used in the same meaning as an approximation index of a differentiation function. Examples thereof include R² value, correlation coefficient, regression coefficient, residual sum of squares (difference in feature from differentiation function), and the like.

As used herein, “differentiation function model” refers to a model of a function used for differentiation of trait or the like. Examples thereof include, but are not limited to, differentiation models with machine learning using a neural network such as multilayer perceptron or CNN.

The learning unit can be configured to divide the genetic information on the plurality of individuals, learn relationships between partial genetic information and trait information, and integrate relationships between a plurality of pieces of partial genetic information and trait information to learn the relationship between the genetic information and the trait information. Such divided learning of genetic information can be effective for dealing with a large amount of genetic information on an individual.

In the present disclosure, an analysis unit analyzes diagnosis of the individual and/or treatment or prophylaxis on the individual from the trait information predicted in the calculation unit. Since trait information is information on a target individual, other information (e.g., disease information database or the like) can be referred to diagnose or assist in the diagnosis of a disease, symptom, or the like from which the individual is suffering or is potentially suffering. A suitable treatment method or dosing information can be computed or suggested by referring to other information (e.g., a disease information database, a drug information database, or the like) in accordance with the result of diagnosis.

In the present disclosure, a display unit displays the trait information predicted in the calculation unit. Anything can be used as the display unit, as long as a user can perceive the trait prediction result. A television, smartphone or tablet screen, monitor, sound generator (e.g., speaker), or the like can be used. Such a display can appropriately display selected items among the calculation result predicted at the calculation unit. Examples of such displayed items include, but are not limited to, recommendation of the optimal anticancer agent for the cancer of the patient and recommendation of the optimal treatment plan for the treatment of the disease of the patient.

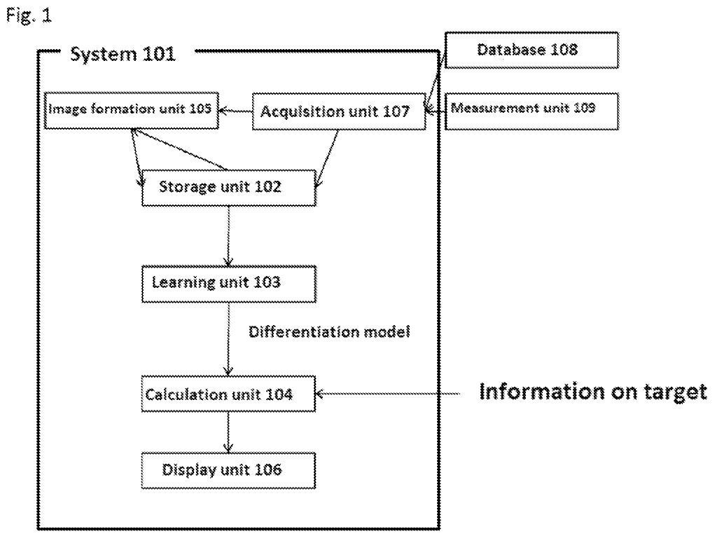

The detailed operation of system 101 of the present disclosure is described with reference to FIG. 1, solely for the purpose of exemplification. The system 101 has an acquisition unit 107. Data used for learning is acquired by the acquisition unit 107 and stored in a storage unit 102. As data for learning, data in an existing database 108 can be acquired (downloaded), or data can be acquired from a measurement unit 109 comprising an instrument for measuring information on an individual.

The system 101 can optionally comprise an image formation unit 105 for forming an image of genetic information on an individual. In an embodiment that has an image formation unit, the system can directly store acquired information in the storage unit 102, then transmit genetic information to the image formation unit 105, form an image of the information, and store the information again. Alternatively, the system can transmit information obtained by the acquisition unit 107 to an image formation unit, form an image, and store the image in a storage unit. The system 101 can optionally perform these operations in combination. Specifically, information derived from each of the plurality of individuals is not necessarily stored by the same process.

A differentiation model is generated by learning at a learning unit 103 based on genetic information and trait information on a plurality of individuals stored in the storage unit. Trait information on a subject is predicted at the calculation unit 104 based on information on the subject (e.g., genetic information) using the generated differentiation model. The predicted result can be displayed on a display unit 106. Data can be stored at any point during the operation of the system 101.

(Embodiment Using the Cloud, IoT, and AI)

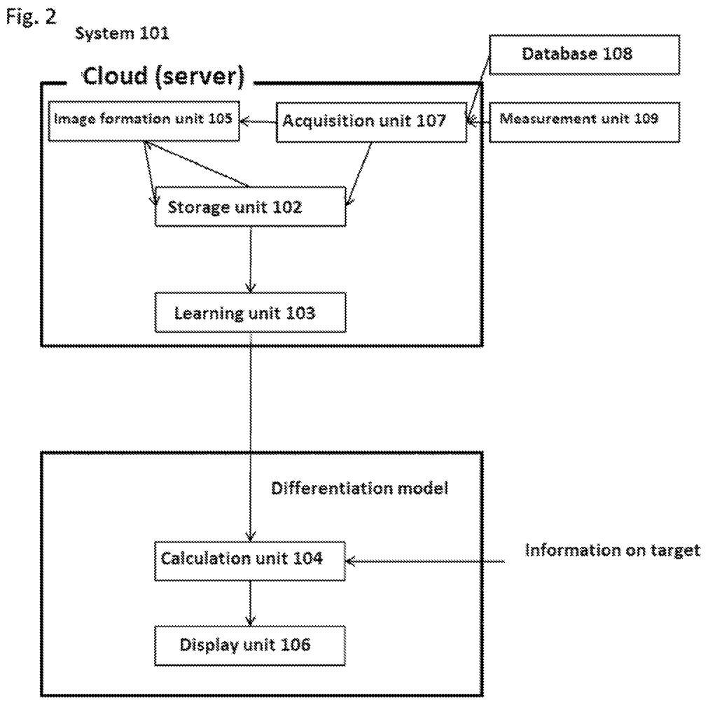

The trait prediction technology of the present disclosure can be provided in a form comprising all components as a single system 101 or apparatus (see FIG. 1). Alternatively, an embodiment of a trait prediction apparatus, which mainly receives an input of genetic information on an individual and displays the result while performing calculation including those for a differentiation model on a server or cloud, can also be envisioned (see FIG. 2). A portion or all of the technology can be implemented using IoT (Internet of Things) and/or artificial intelligence (AI). Alternatively, a semi-standalone embodiment, wherein a differentiation model is stored in a trait prediction apparatus to perform differentiation therein, but major calculation such as calculation of a differentiation model is performed in a server or cloud, can also be envisioned (FIG. 2). Since data cannot always be transmitted/received at some locations such as hospitals, a model that can also be used without connection is envisioned. A system for generating a differentiation model comprising up to a learning unit and a prediction system storing and utilizing an obtained differentiation model in a calculation unit are also embodiments of the present disclosure (FIG. 2). “Software as a service (SaaS)” mostly falls under such a cloud service. It is also possible to provide a contractor service, which distributes a program for forming an image of patient data, asks the patient to transmit only data converted into an image at a deployed location such as a hospital, and receives and analyzes the data.

Anything can be used as the display unit, as long as a user can perceive the trait prediction result. An input/output device, display device, television, monitor, sound generator (e.g., speaker), or the like can be used.

A preferred embodiment can comprise a function for improving a differentiation model. Such a function can be in a learning unit or comprised as a separate module. Such a function for improving a differentiation model can comprise options such as option 1 (period of 1 year at once or twice a year), optional 2 (period of 1 year at once every one or two months), option 3 (extended period at once or twice a year), and option 4 (extended period at once every one or two months).

Data can also be stored as needed. While data is generally stored on the server side (FIG. 2), data can be stored on the terminal side for not only fully equipped models, but also for cloud models (not shown because such an embodiment is optional). When service is provided on the cloud, data storage options such as standard (e.g., up to 10 GB on the cloud), option 1 (e.g., increased to 1 TB on the cloud), option 2 (divided and stored on the cloud by setting a parameter), and option 3 (stored on the cloud by differentiation model) can be provided. Data can be stored to create bid data in a storage unit by pulling in data from all sold apparatuses in order to continuously update a differentiation model, or construct a new model to provide a new differentiation model software. The storage unit can be a recording medium such as CD-R, DVD, Blu-ray, USB, SSD, or hard disk, or the storage unit can be stored in a server or configured in a form that appropriately records on the cloud.

The present disclosure can have a data analysis option, which can provide classification of patterns for a patient (search for a patient cluster based on a change in the pattern of a feature or differentiation accuracy) or the like. Specifically, such an option is envisioned as an option in the calculation method of the calculation unit 104.

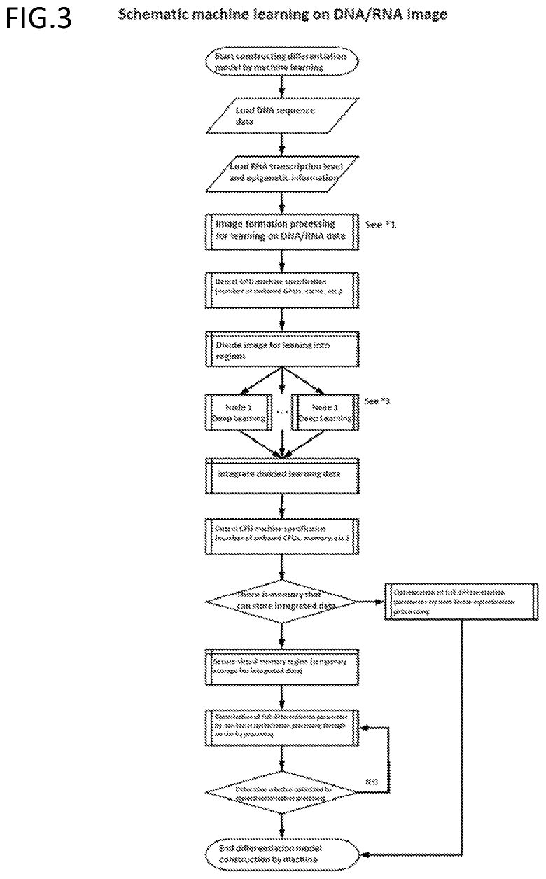

An example of the differentiation model construction of the present disclosure when using DNA data or RNA data as genetic information is described in more detail with reference to FIG. 3. The description is intended for exemplification, not limitation.

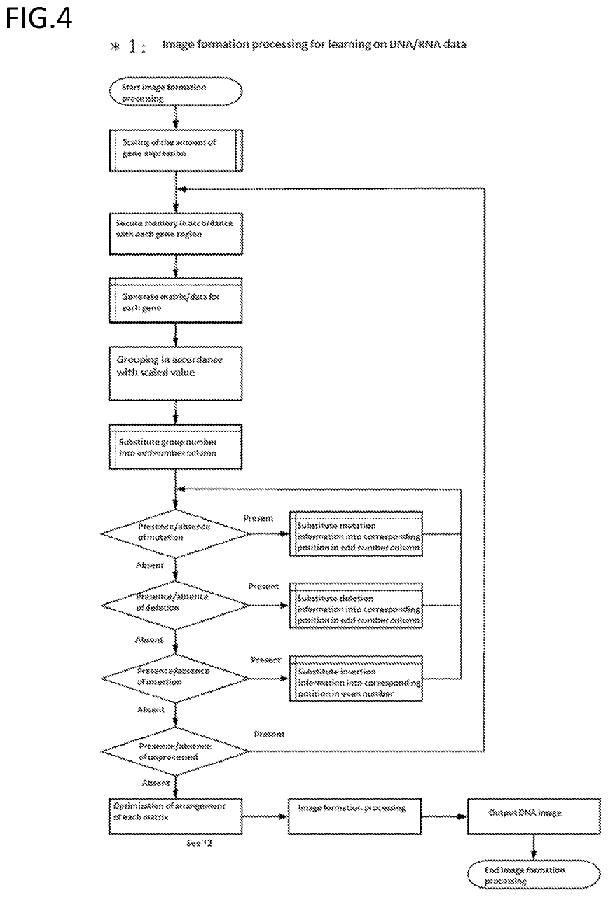

First, DNA sequence data is loaded, then RNA transcription level and epigenetic information are loaded. This can also be performed using the acquisition unit 107 in the system 101. Next, the DNA and RNA data is subjected to processing to form an image for learning. As the image formation method, the image formation method described in detail elsewhere herein with reference to FIG. 4 can be employed.

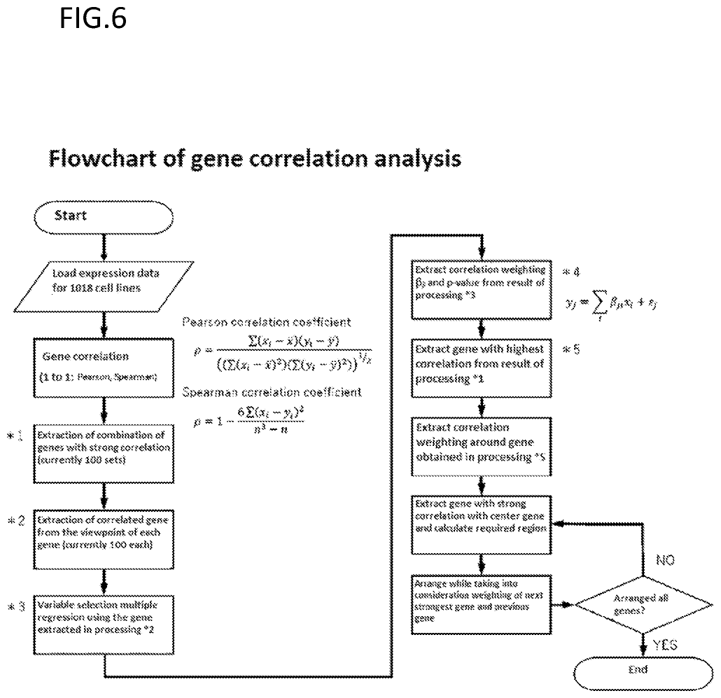

During learning, the DPU machine specification (number of onboard GPUs, cache, etc.) is detected. An image used for learning is divided into regions based on the result of detection. The divided image is studied at each node. For details of divided learning, the divided learning method described in detail elsewhere herein with reference to FIG. 6 can be employed. Divided learning data is then integrated. The CPU machine specification (number of onboard CPUs, memory, etc.) is detected upon integration of data. If there is memory that can store the integrated data, a full differentiation parameter is optimized by non-linear optimization processing to construct a differentiation model. If there is no memory that can store the integrated data, a virtual memory region is secured, and the integrated data is temporarily stored. A full differentiation parameter is then optimized by non-linear optimization processing through on the fly processing. Differentiation is then performed as to whether data is optimized by divided optimization processing. If not optimized, non-linear optimization processing is performed again through on the fly processing to perform another differentiation. When differentiated as optimized, differentiation model construction is ended.

(Image Formation Method)

One aspect of the present disclosure is a method of forming an image of genetic information. In one embodiment, image formation can be understood as comprising the step of generating image data having a plurality of pixels, each of which comprising position information and color information. Such image data can have data for genetic information stored. One feature of the image formation method of the present disclosure can be formation of an image of sequence data for a genetic factor population comprising a plurality of genetic factors and expression data for a genetic factor population comprising a plurality of genetic factors. Such image formation can be advantageous in terms of enabling simultaneously learning of sequence information and expression information. In addition, it is a well-known fact that recent deep learning has significantly improved image recognition performance as compared to conventional machine learning methods and is applied in various fields. Thus, it is understood that current deep learning methods can be efficiently used for any data converted into an image.

One embodiment of the present disclosure is a method of forming an image of sequence data for a genetic factor population comprising a plurality of genetic factors and expression data for a genetic factor population comprising a plurality of genetic factors, comprising the step of: generating image data for storing the sequence data for the genetic factor population and the expression data for the genetic factor population, the image data having a plurality of pixels, each of which comprising position information and color information. In another embodiment of the present disclosure, each of the plurality of genetic factors is associated with a region in the image data, and the step of generating the image data can comprise the step of: converting an amount of expression of the genetic factor into color information in a certain region within a region associated with the genetic factor and/or information on an area of a region having a certain color in the region.

In one embodiment, data associated with the amount of expression, when converted into an image, can be grouped into a certain number of levels. The actual amount of gene expression varies significantly for each gene. The standard deviation of the expression distribution thereof also varies significantly. Thus, colors required for image formation would be high if data for amount of expression is directly used in learning. In addition, the meaning of a change in the amount of expression of the same value would be different among genes. Thus, the amounts of expression can be scaled so that the standard deviation would be constant (e.g., 1) from data for a large number (e.g., over 1000) of samples. Furthermore, expression amount values changed in this manner can be coarse grained by grouping. This can be advantageous for improving efficiency of learning and reducing the data size in machine learning.

Since the meaning of coarse graining would be lost if the unit scale of coarse graining by grouping is too fine, the unit scale can be gradually changed, little by little, for genes with the smallest standard deviation (actual standard deviation is 1 or less) in the data as of loading to determine the final unit scale in a range where normal distribution approximation is deemed effective. The expression amounts can be scaled into a group of about 120 to about 180 levels, about 130 to about 160 levels, or about 150 levels. A monochrome image can also be used as an image. For monochrome images, color information at each position would be just the brightness value. While the levels thereof are not particularly limited, monochrome images with, for example, 256 levels of brightness can be used. This can lead to efficient data compression. Further, information on mutation, deletion, and insertion, which are very small pieces of information as a pixel region, can be made conspicuous by expressing with colors having lower brightness compared to discrimination used in amounts of expression (e.g., discrimination with 150 levels of brightness). A, T, G, and C bases can also be expressed by 10 different levels of brightness for more clear discrimination. Such required brightness level setting is understood as the optimal setting in terms of both data compression and improvement in efficiency of learning in relation to the image formation method of the present disclosure and is the significant difference from conventional art.

In one embodiment of the present disclosure, it is understood that the purpose of image formation is to express gene expression amount or mutation information using the difference in the positions and brightness of color in a two dimensional image region, which can compress data without losing the amount of information to about 1/24 (about 400 [MB]) compared to numerical data (about 9.6 [GB]) by converting to a compressed image format such as JPG or PNG. It is understood that such image formation is advantageous in terms of not only compression of data size, but also in allowing application to conventional methods by converting numerical data into two dimensional position information or saturation information.

Sequence data for a genetic factor population can comprise sequence data for a factor associated with an event that propagates a genetic trait from a parent cell to a daughter cell. Such a factor is, for example, a DNA sequence.

Examples thereof include a gene encoding a protein, exon sequence, intron sequence, regulatory region sequence, and the like. Expression data for a genetic factor population can comprise expression data for a factor associated with information transmission for only the current generation. Such expression data for a factor is, for example, expression data for RNA. Examples thereof include amounts of expression of mRNA, miRNA, siRNA, and lnRNA and the like.

Sequence data and expression data formed into an image can be data for a genetic factor of the same individual.

Sequence data for a genetic factor population can comprise a sequence of a certain region on a genomic DNA. For example, sequence data for a genetic factor population can comprise a DNA sequence encoding a sequence of a gene on a genomic DNA, an exon sequence of a gene on a genomic DNA, and/or a non-coding RNA on a genomic DNA.

An image of sequence information can be formed by converting information on the position and type of a mutation in a sequence of a certain genetic factor into position and color information within a region associated with the genetic factor. Specifically, instead of reflecting each of all sequence information in an image, only information on a portion with a mutation can be reflected in an image. This can lead to reduction in the amount of information.

Information on a modification on a sequence can also be reflected in an image. This can be performed by a step of converting information on a modification in a sequence of a certain genetic factor into position and color information within a region associated with the genetic factor.

Expression data can comprise expression data for a transcription unit. For example, expression data for mRNA can comprise data for an amount of expression, splicing, a transcription start point, and/or an epigenetic modification of mRNA. Expression data for a genetic factor population can comprise expression data for an miRNA, an snoRNA, an siRNA, a tRNA, an rRNA, an mitRNA, and/or a long chain non-coding RNA. Expression data for a genetic factor population can comprise data for an amount of expression, splicing, a transcription start point, and/or an epigenetic modification of an miRNA, an snoRNA, an siRNA, a tRNA, an rRNA, an mitRNA, and/or a long chain non-coding RNA.

Each of a plurality of genetic factors can be associated with a region in image data, and the amount of expression of a genetic factor can be converted to color information in a certain region within a region associated with the genetic factor and information on an area of a region having a certain color in the region.

When a genetic factor comprises an exon, an amount of expression of a transcript corresponding to the exon or a portion thereof can be converted to color information in a certain region within a region associated with the exon and/or information on an area of a region having a certain color in the region to store splicing and/or transcription start point of the genetic factor in image data.

When a genetic factor comprises one or more genes, each of the one or more genes is associated with a region in image data, and sequence and expression information for the gene can be stored in the image data by the steps of converting information on a position and type of a mutation in a genomic sequence of a certain gene into position and color information within a region associated with the gene, and converting an amount of expression of mRNA transcribed from the gene into color information in a certain region within a region associated with the gene and/or information on an area of a region having a certain color in the region.

When a genetic factor comprises one or more DNA sequences encoding non-coding RNA, each of the one or more DNA sequences is associated with a region in image data, and sequence and expression information on the non-coding RNA can be stored in the image data by the steps of converting information on a position and type of a mutation and/or epigenetic modification in a genomic sequence of a DNA sequence encoding a non-coding RNA into position and color information within a region associated with the gene, and converting information on an amount of expression, splicing, transcription start point, and epigenetic modification of non-coding RNA transcribed from the DNA sequence into position and color information within a region associated with the gene.

When a genetic factor comprises one or more DNA sequences of a non-expression region and one or more transcription units, each of the one or more DNA sequences and transcription units is associated with a region in image data, and information on a sequence of the non-expression region and expression associated therewith can be stored in the image data by the steps of converting information on a position and type of a mutation and/or epigenetic modification in a genomic sequence of a DNA sequence into position and color information within a region associated with the gene, and converting information on expression of the transcription unit into position and color information in a certain region within a region associated with the transcription unit.

When a genetic factor comprises one or more DNA sequences and transcription units on a genome, each of the one or more DNA sequences and transcription units on a genome is associated with a region in image data, and information on a sequence and expression associated therewith can be stored in the image data by the steps of converting information on a position and type of an epigenetic modification in a genomic sequence of a DNA sequence into position and color information within a region associated with the DNA region, and converting information on expression of a transcription unit into position and color information in a certain region within a region associated with the transcription unit.

As sequence information, a factor associated with an event that propagates a genetic trait from a parent cell to a daughter cell in the nucleus or mitochondria under the control of an RNA polymerase, which is a DNA sequence encoding not only a coding RNA or mRNA encoding a protein, but also miRNA, snoRNA, siRNA, tRNA, rRNA, or mitRNA with a relatively short strand up to 10s of bases, as well as longer chain non-coding RNA as non-coding RNA can be targeted in the image formation of the present disclosure. A DNA sequence of a non-expression region away from a complimentary portion of the expression product described above as well as epigenetic modification on a DNA or the like can also be targeted.

As expression information, a DNA sequence encoding not only a coding RNA or mRNA encoding a protein, but also miRNA, snoRNA, siRNA, tRNA, rRNA, or mitRNA with a relatively short strand up to 10s of bases as well as longer chain non-coding RNA as non-coding RNA, under the control of an RNA polymerase, including a genetic factor (an amount of expression, splicing, a transcription start point, an epigenetic modification, and the like of transcription unit (RNA and miRNA)) can be targeted.

This consolidates comprehensive information related to sequences and comprehensive information related to expression in a single image. Mutations in a region where a function is not identified are possibly associated with a trait such as anticancer agent sensitivity.

For example, by forming an image of information on amounts of expression of various RNA with a genomic genetic sequence, information on a sequence of a gene and an amount of expression of the gene can be consolidated into a single region to simultaneously process information on a sequence of a gene, an amount of expression of the gene, etc.

When targeting mRNA, a somatic cell mutation, embryonic cell mutation, genetic polymorphism, and changes to a minor base other than A, T, G, and C (e.g., measured by a nanopore sequencer) can also be reflected in an image as a base substitution of a gene. As gene expression, not only the mean expression amount of the entire gene as an expression unit, but also a change in splicing (including alternative, splice-out, etc.) or transcription start point by tissue/cell (e.g., such sequence information can be obtained using RIKEN FANTOM) can be reflected. Methylated C5, A1, A5, phosphorylation, or the like can also be reflected as an epigenomic or epitranscriptome modification.

With regard to non-expression regions, opening/closing of chromatin is involved with a transcription event into an RNA almost without exception, so that the entire genome can be profiled by immunoprecipitation-sequencing or the target can be narrowed down and analyzed by immunoprecipitation-PCR. For example, modifications of trimethyl me3 (three methyl groups) and dimethyl me2 (two methyl groups) of histone H3 lysine 4 (H3K4) open nearby chromatin, promote recruitment of transcription factor thereto, and act to activate transcription. Methylation of H3K9 (me3 or me2) acts to close chromatin and suppress transcription. Transcription can be mapped by analysis thereof by immunoprecipitation-sequencing or immunoprecipitation-PCR. It is understood that transcription activity of a region between genes can be seen by including such information.

Another embodiment of the present disclosure can provide a method for creating a model for predicting trait information on an individual from sequence information and expression information on a genetic factor of the individual. The method can comprise the steps of: forming an image of sequence information and expression information on a genetic factor of a plurality of individuals by the method described elsewhere herein to provide image data; providing trait information on the plurality of individuals; and extracting an expression of a feature in an image correlated with a trait from the image data and the trait information by deep learning.

While the process of image formation can be described in more details with reference to FIG. 4, the description is not intended for limitation. The amount of gene expression is scaled in image formation processing. A memory in accordance with each gene region is then secured. In addition, a data matrix of each gene is created. The amounts are grouped in accordance with scaled values, and the group numbers are substituted into an odd number column of the matrix.

When the presence/absence of a mutation (sequence substitution) is differentiated and a mutation is found, mutation information is substituted into a corresponding position in an odd number column. When the presence/absence of a deletion is differentiated and a deletion is found, deletion information is substituted into a corresponding position in an odd number column. When the presence/absence of an insertion is differentiated and an insertion is found, insertion information is substituted into a corresponding position in an even number column. With no more unprocessed data, the arrangement of each matrix is optimized to perform image formation processing. The arrangement can be optimized in accordance with the procedure described below. An image is outputted, and the processing ends.

(Arrangement Optimization)

Some aspects of the present disclosure are directed to optimization of the arrangement of genetic factors in image formation. The arrangement of genetic factors on an image is not particularly limited. For example, genetic factors can be lined up in the order of description in a database or in accordance with some type of numbers. However, further improvement in the efficiency of machine learning using an image can be expected by optimizing the arrangement of genes. Thus, optimization of the arrangement of genetic factors according to some aspects of the present disclosure can be applied for the purpose of such improvement. In particular, it is understood that the efficiency of machine learning using an image can be improved if a genetic factor with high external correlation contribution is arranged in the middle, and genetic factors are arranged therearound in the order of greater correlation weighting.

Thus, this aspect of the present disclosure provides a method of forming an image of genetic information, the genetic information containing sequence data and/or expression data for a genetic factor population comprising a plurality of genetic factors, the method comprising the steps of: generating image data for storing the sequence data and/or expression data for the genetic factor population, the image data having a plurality of pixels, each of which comprising position information and color information, wherein the step comprises associating each of the plurality of genetic factors with a region in the image data, and regions associated with each genetic factor are arranged so that those with a high correlation weighting of each genetic factor are in proximity.

The step of generating the image data can comprise computing an area of a region in image data that is required for the genetic factor. For example, the area of a region that is required can be computed in accordance with the size (sequence length) of sequence information on a genetic factor.

The correlation weighting of genetic factors can be computed by: extracting a combination of genetic factors with a strong correlation from correlation analysis between genetic factors; extracting a genetic factor with a strong correlation for each of the genetic factors; performing variable selection multiple regression using the extracted genetic factors, and computing a correlation weighting from a result of the variable selection multiple regression.

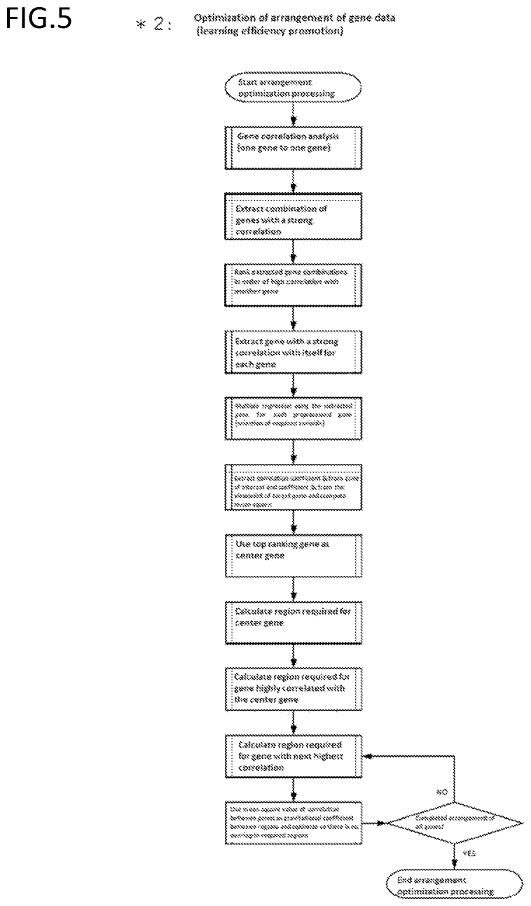

Optimization of arrangement is described in further detail with reference to FIG. 5, which is not intended for limitation. Gene correlation analysis is performed in optimization of arrangement (see FIG. 6). A combination of genes with a strong correlation is extracted. Ranking is determined in the order of high correlation with another gene in the extracted combination of genes. For each gene, a gene with a strong correlation with itself is extracted. Multiple regression (selection of required variable) using the extracted gene is performed for each preprocessed gene. Correlation coefficient βⱼᵢ from a gene of interest and coefficient βᵢⱼ viewed from a target gene are extracted, and the mean square is computed. The top ranked gene is used as the center gene. The region that is required for the center gene is computed. The region that is required for a gene which is highly correlated with the center gene is computed. The region that is required for the next highly correlated gene is computed. The mean square value of correlation between genes is used as the gravitational coefficient between regions for optimization that would not result in overlap in regions that are required. It is determined whether arrangement of all genes has been completed. If not completed, the processing described above is repeated. When arrangement of all genes is completed, arrangement optimization processing is ended.

Gene correlation analysis is described in more detail with reference to FIG. 6. Expression data for a plurality of individuals (e.g., 1018 cell lines) is loaded, and gene correlation analysis is performed. 1-to-1 correlation analysis is performed using a Pearson correlation coefficient

$$ρ = \frac{\sum ( {x}_{i} - \overline{x} ) ( {y}_{i} - \overline{y} )}{{( ( \sum {( {x}_{i} - \overline{x} )}^{2} ) ( \sum {( {y}_{i} - \overline{y} )}^{2} ) )}^{1 / 2}} , \qquad [ Numeral 1 ]$$

or
A Spearman's correlation coefficient

$$ρ = 1 - \frac{6 \sum {( {x}_{i} - {y}_{i} )}^{2}}{{n}^{3} - n} . \qquad [ Numeral 2 ]$$

A combination of genes with a strong correlation is subsequently extracted. In addition, a correlated gene from the viewpoint of each gene is extracted. Variable selection multiple regression is performed using the gene extracted by such processing. Correlation weighting Bⱼᵢ and p-value are extracted from the result of multiple regression. Correlation weighting Bⱼᵢ can be computed as a value satisfying

$${y}_{j} = {\sum }_{i} {β}_{ji} {x}_{i} + {ɛ}_{j} . \qquad [ Numeral 3 ]$$

A gene with the greatest correlation is extracted from the result of extracting a combination of genes with a strong correlation. Correlation weighting is extracted with the gene obtained by this processing at the center. A gene with a strong correlation with the center gene is then extracted, and the required region is calculated. Genes are then arranged while taking into consideration the weighting of the next strongest gene and the previous gene. It is determined whether all genes have been arranged. If not completed, the processing described above is repeated. Arrangement optimization processing is ended when arrangement of all genes have been completed.

The arrangement of genetic factors can be optimized as a MinSum problem (minimization problem of arrangement distance). While some are formulated as a city facility location problem, the optimization of the arrangement of genetic factors of the present disclosure is different from a facility location problem in that (1) ends of a region of an effective range (areas of genetic factors in this case) are in contact with each other when arranged, and (2) facility distance (distance between centers in this case) is not necessarily proportional to the user/degree of importance (weighting and significance in this case).

(Data Structure)

Another aspect of the present disclosure is directed to a specific data structure of image data. An embodiment of the present disclosure provides, for example, a data structure of image data representing sequence information on a genetic factor population comprising a plurality of genetic factors and expression information on a genetic factor population comprising a plurality of genetic factors, wherein the image data has a plurality of regions associated with the plurality of genetic factors; each position in a sequence of a genetic factor is associated with a position within the regions associated with the genetic factor; information on a substitution, a deletion, and/or an insertion at each position in the sequence of the genetic factor is stored as color information at a position associated with the position; and expression data for the genetic factor is stored as color information at a certain region in the regions, and/or information on an area of a region having a certain color in the region.

Information on an epigenetic modification at each position in a sequence of the genetic factors can be further stored as color information at a position associated with the position. For example, methylation at each position in a sequence of an miRNA in the plurality of genetic factors can be stored as color information at a position associated with the position. The image data can be a matrix having a row and a column, and each of the positions can be stored as a combination of a row and a column.

Sequence information can comprise a DNA sequence of a region on a genome. Examples thereof include a gene, an exon, an intron, a non-expression region, and/or a non-coding RNA encoding region.

Expression information can comprise information on an amount of expression, splicing, a transcription start point, and/or an epigenetic modification of a transcription unit selected from the group consisting of an mRNA, an miRNA, an snoRNA, an siRNA, a tRNA, an rRNA, an mitRNA, and/or a long chain non-coding RNA.

Image data can have a plurality of regions associated with a region and/or transcription unit on each genome. A region associated with a region on a genome can consist of a number of columns dependent on the length of the region on a genome and a constant number of rows. Each position in a sequence on a region on the genome can be associated with a position in an odd number column within the region associated with the region on the genome. Information on a substitution, a deletion, and/or an insertion at each position in the sequence of a region on the genome can be stored as color information at a position in an odd number column associated with the position. The color information can be color information indicating the absence of a mutation, color information indicating a substitution with A, color information indicating a substitution with T, color information indicating a substitution with G, color information indicating a substitution with C, color information indicating the presence of a deletion, or color information indicating the presence of an insertion adjacent to the position. Color information indicating an inserted sequence can be stored as information on an inserted sequence, with a position in an even number column adjacent to a position having color information indicating the presence of an insertion as a starting point.

Information on an epigenetic modification at each position in the sequence of a region on the genome can be stored as color information at a position in an odd number column associated with the position. The color information can comprise color information indicating the absence of an epigenetic modification, color information indicating DNA methylation, color information indicating histone methylation, color information indicating histone acetylation, color information indicating histone ubiquitination, color information indicating histone phosphorylation, or the like.

An amount of expression of a transcription unit transcribed from a region on a genome can be stored as a shade of a color in a region in an image associated with the region on the genome and/or information on an area of a region having a certain color in the region.

An amount of expression of an mRNA associated with a gene for a region on a genome that is the gene can be stored as a shade of a color in a certain region in the region and/or information on an area of a region having a certain color in the region.

The image formation method and image data described above are useful in comprehensive handling of genetic information on an individual, which are useful in any technical field related to organisms such as the medical, agricultural, animal husbandry, food, environmental, and pharmaceutical (drug development and postmarketing surveillance) fields.

(Divided Learning)

Another aspect of the present disclosure provides a method for creating a model for predicting a relationship between an image and information associated with the image. One of the features of the method can be dividing an image for learning. The method can comprise the steps of: providing a set of a plurality of images and a plurality of pieces of information associated with the plurality of images; obtaining a plurality of divided learning data by dividing the plurality of images and learning a relationship between a portion of the plurality of images and information associated with the images; and integrating the plurality of divided learning data to generate a model for predicting the relationship between the image and the information associated with the image.

The integration step can comprise detecting a GPU specification and a CPU specification comprising an amount of on-board memory using a CPU machine with a GPU installed therein. The integration step can comprise optimizing a non-linear optimization processing algorithm that can utilize a Read-Write file on an HDD and utilize a CPU memory as much as possible.

The non-linear optimization processing algorithm can be an algorithm capable of calculation independent of data size by transferring required data to a memory as needed to perform calculation, and returning a calculation result to an HDD (on the fly memory processing). The non-linear optimization processing can comprise optimizing a full differentiation parameter.

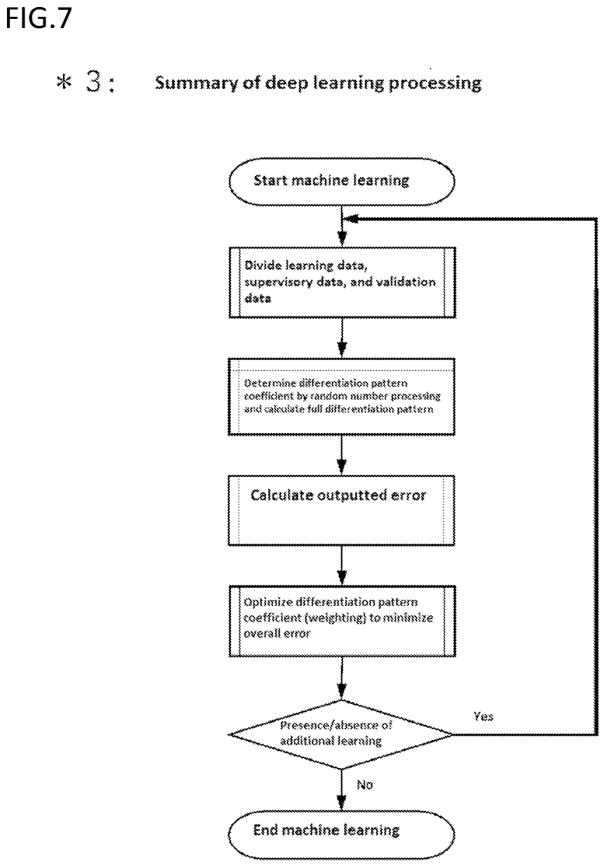

The divided image learning is described in more detail with reference to FIG. 7, without an intention of limitation. Machine learning can be performed by deep learning processing. For machine learning, learning data, supervisory data, and validation data are divided. A differentiation pattern coefficient is determined by random number processing, and a full differentiation pattern is calculated. The outputted error is calculated. The differentiation pattern coefficient (weighting) is optimized so that the overall error is minimized. Presence/absence of additional learning is determined. When addition learning is required, the processing described above is repeated. If additional learning is not required, machine learning is ended.

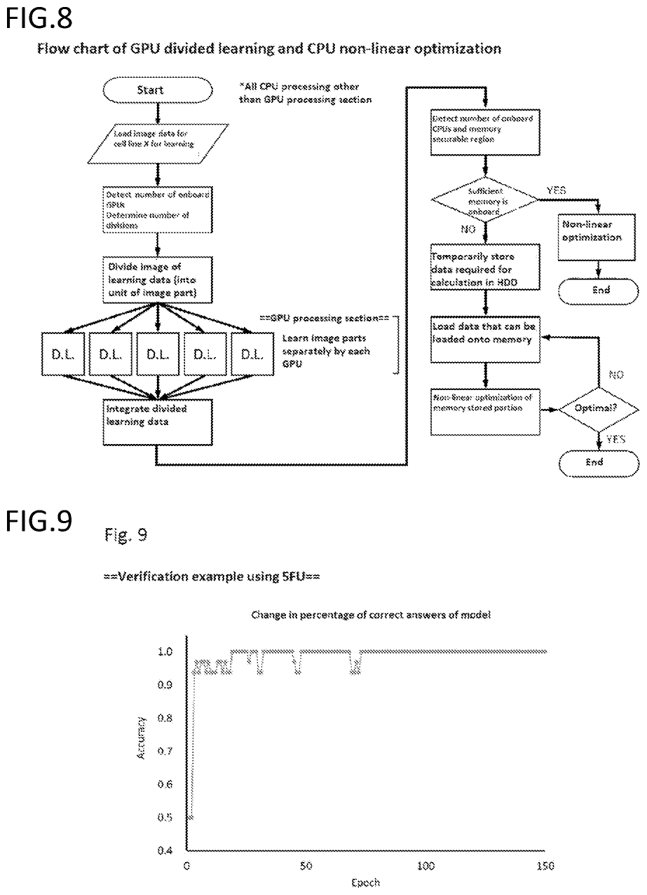

The flow of learning including integration of divided learning data is described in further detail with reference to FIG. 8, without an intention of limitation. Image data for learning is loaded. The number of onboard GPUs is detected to determine the number of divisions. An image of learning data is divided. Different image sites can be learned by each GPU at the GPU processing section. Each node in learning can be physically separated or integrated. Divided learning data is integrated. The number of onboard CPUs and memory securable regions are detected. If sufficient memory is onboard, non-linear optimization is performed, and the processing is ended. If sufficient memory is not onboard, data required for calculation is temporarily stored in an HDD, and only data that can be loaded into memory is loaded. Non-linear optimization is performed on the data stored in memory. It is determined whether it is optimized. If not optimized, processing is repeated. If it is determined to be optimized, processing is ended.

The method of divided learning described above improves the efficiency in machine learning using a relatively large sized data (e.g., image data). For example, the method is useful in learning using biological information formed into an image as well as learning in fields with a large amount of data such as physics and astronomy and learning in object recognition, character recognition, or the like.

The ability to differentiate each divided learning data can be verified in divided learning. For images, the correlation with a response variable such as trait information can be verified for each region from dividing an image. The ability to differentiate and/or correlation can be verified by subjecting the relationship between each region and response variable to machine learning and determining whether the predictive ability converges when the number of epochs is increased. The overall learning efficiency can be improved by selecting and then integrating divided learning data with an ability to differentiate from each divided learning data. Alternatively, divided learning data with an ability to differentiate can be selected from each divided learning data to use the data itself as a prediction model.

The degree of division can be adjusted in accordance with the overall size. When an image prepared from forming an image of genetic mutation information and expression information is used, the image can be divided into a size that would store information of, for example, about 100 to about 200 genes per region.

As a system, the following can be provided: a system for predicting trait information on an individual, comprising:

a storage unit for storing genetic information on a plurality of individuals and trait information on the plurality of individuals, the genetic information containing sequence information and expression information on a genetic factor;

a learning unit configured to learn a relationship between genetic information and trait information from the genetic information on the plurality of individuals and the trait information on the plurality of individuals by forming an image of the genetic information on the plurality of individuals; and

a calculation unit for predicting trait information on an individual from genetic information on the individual based on the relationship between the genetic information and the trait information;

wherein the learning unit is configured to divide an image generated by forming an image of the genetic information on the plurality of individuals, learn a relationship between each region of the image and trait information, select a region where a model with an ability to differentiate trait information can be generated from each region, and generate a model for predicting trait information from each region on the image.

As a method, the following can be provided: a method for creating a model for predicting a relationship between genetic information containing sequence information and expression information on a genetic factor of an individual and trait information on the individual, comprising the steps of:

providing a set of a plurality of images formed from sequence information and expression information on a genetic factor of a plurality of individuals and a plurality of pieces of trait information associated with the plurality of images;

obtaining a plurality of divided learning data by dividing the plurality of images and learning a relationship between a portion of the plurality of images and the information associated with the images; and

selecting divided learning data with an ability to differentiate trait information from the plurality of divided learning data to generate a model for predicting trait information from each region of the image.

The present disclosure also provides a program causing a computer to execute a method for creating a model for predicting a relationship between genetic information containing sequence information and expression information on a genetic factor of an individual and trait information on the individual, the method comprising the steps of:

providing a set of a plurality of images formed from sequence information and expression information on a genetic factor of a plurality of individuals and a plurality of pieces of trait information associated with the plurality of images;

obtaining a plurality of divided learning data by dividing the plurality of images and learning a relationship between a portion of the plurality of images and information associated with the images; and

selecting divided learning data with an ability to differentiate trait information from the plurality of divided learning data to generate a model for predicting trait information from each region of the image.

When an image is created from genetic information including sequence information and expression information on a genetic factor, it is possible to select a portion of an image from which divided learning data with an ability to differentiate trait information is obtained, determine whether trait information can be predicted based on the expression information from the portion of an image from which divided learning data with an ability to differentiate trait information is obtained, and select a portion from which trait information cannot be predicted based on the expression information. This enables use as a method of identifying a gene correlated with a trait or a mutation thereof. From a gene contained at a portion from which trait information cannot be predicted based on expression information, a gene having a mutation that correlates with trait information can be identified. Such a gene or a mutation thereof is possibly functionally correlated with a trait. It is understood that the identified gene can be used in the prediction of trait information on an individual. The identified gene can itself be a model for predicting trait information on an individual, and optionally can be used by integrating the gene into a model for predicting trait information on an individual.

For a certain region, whether trait information can be predicted based on expression information can be determined by, for example, cluster analysis on the amount of expression of a gene contained in the region for each individual. This can also be determined using any regression analysis or machine learning method besides cluster analysis.

As a system, the following can be provided: a system for predicting trait information on an individual, comprising:

a storage unit for storing genetic information on a plurality of individuals and trait information on the plurality of individuals, the genetic information containing sequence information and expression information on a genetic factor;

a learning unit configured to learn a relationship between genetic information and trait information from the genetic information on the plurality of individuals and the trait information on the plurality of individuals by forming an image of the genetic information on the plurality of individuals; and

a calculation unit for predicting trait information on an individual from genetic information on the individual based on the relationship between the genetic information and the trait information;

wherein the learning unit is configured to divide an image generated by forming an image of the genetic information on the plurality of individuals, learn a relationship between each region of the image and trait information, select a region where a model with an ability to differentiate trait information can be generated from each region, determine whether trait information can be predicted based on expression information in each region, and identify a gene having a mutation that is correlated with trait information from a gene in a region where trait information cannot be predicted based on expression information, and

the calculation unit is configured to predict the trait information on the individual based on information on the gene having a mutation that is correlated with the trait information.

As a method, the following can be provided: a method for identifying a mutation of a gene associated with a trait, comprising the steps of:

providing a set of a plurality of images formed from sequence information and expression information on a genetic factor of a plurality of individuals and a plurality of pieces of trait information associated with the plurality of images;

obtaining a plurality of divided learning data by dividing the plurality of images and learning a relationship between a portion of the plurality of images and information associated with the images; and

selecting a portion of an image where divided learning data with an ability to differentiate trait information can be obtained;

determining whether trait information can be predicted based on expression information from the portion of an image where divided learning data with an ability to differentiate trait information can be obtained to select a portion where trait information cannot be predicted based on expression information; and

identifying a gene having a mutation that is correlated with trait information from a gene contained at the portion where trait information cannot be predicted based on expression information.

Even when convergent and isolatable from only the amount of gene expression, a gene that can be important for differentiation can be extracted by dividing an image of a specific region further. A region that is convergent and differentiable from only information on the amount of gene expression even in a region of a divided image is genetic information that is important for differentiation. Thus, genetic information can be extracted by repeated division.

Even when it is not isolatable from only the amount of gene expression despite being convergent, information on a genetic mutation that is important for differentiation can be extracted by further dividing an image of the specific region. Even in such cases, a region that cannot be divided with only information on the amount of gene expression, despite being convergent, is narrowed down, and information on genetic mutations contained in the narrowed down region is extracted.

The present disclosure also provides a program causing a computer to execute a method for identifying a mutation of a gene associated with a trait, the method comprising the steps of:

providing a set of a plurality of images formed from sequence information and expression information on a genetic factor of a plurality of individuals and a plurality of pieces of trait information associated with the plurality of images;

obtaining a plurality of divided learning data by dividing the plurality of images and learning a relationship between a portion of the plurality of images and information associated with the images;

selecting a portion of an image where divided learning data with an ability to differentiate trait information can be obtained;

determining whether trait information can be predicted based on expression information from the portion of an image where divided learning data with an ability to differentiate trait information can be obtained to select a portion where trait information cannot be predicted based on expression information; and

identifying a gene having a mutation that is correlated with trait information from a gene contained at the portion where trait information cannot be predicted based on expression information.

### OTHER EMBODIMENTS

Trait prediction methods according to one or more aspects of the present disclosure have been described based on the embodiments, but the present disclosure is not limited to such embodiments. Various modifications applied to the present embodiments and embodiments constructed by combining constituent elements in different embodiments that are conceivable to those skilled in the art are also encompassed within the scope of one or more aspects of the present disclosure, as long as such embodiments do not deviate from the intent of the present disclosures.

A trait prediction method can be executed by a program. Specifically, the following can be provided: a program causing a computer to execute a method for predicting trait information on an individual, the method comprising: an information providing step for providing genetic information on a plurality of individuals and trait information on the plurality of individuals, the genetic information containing at least two types of information; a learning step for learning a relationship between genetic information and trait information from the genetic information on the plurality of individuals and the trait information on the plurality of individuals; and a predicting step for predicting trait information on an individual from genetic information on the individual based on the relationship between the genetic information and the trait information. The program can further comprise a displaying step for displaying the predicted trait information. A recording medium storing such a program can also be provided.

A system can comprise a program causing a computer to execute a method described herein. For example, the system can comprise a recording medium storing such a program. The system can also comprise a computation apparatus (e.g., computer) for executing an instruction given by a program. A computation apparatus can be physically integrated, or consist of a plurality of constituent elements that are physically separated. The computation apparatus can internally comprise a function corresponding to the image formation unit 105, learning unit 103, calculation unit 104, acquisition unit 107, and the like in the present disclosure as needed.

The system of the present disclosure can be materialized as an ultra-multifunctional LSI manufactured by integrating a plurality of components on a single chip. Specifically, the system can be a computer system comprised of a microprocessor, ROM (read only memory), RAM (random access memory), and the like. A computer program is stored in the ROM. A system LSI can accomplish its function by operating the microprocessor in accordance with the computer program.

The system was referred to as system LSI, but may also be referred to as IC, LSI, super LSI, or ultra LSI depending on the difference in the degree of integration. The approach of building an integrated circuit is not limited to LSI. The system can be materialized with a dedicated circuit or a generic processor. After the manufacture of LSI, a programmable FPGA (Field Programmable Gate Array) or reconfigurable processor which allows reconfiguration of the connection or setting of circuit cells inside the LSI can be utilized.

If a technology of integrated circuits that replaces LSI by advances in semiconductor technologies or other derivative technologies becomes available, functional blocks can obviously be integrated using such technologies. Application of biotechnology or the like is also a possibility.

One embodiment of the present disclosure can be not only such an image formation analysis, diagnosis, treatment, prevention prediction apparatus, but also a test analysis/diagnosis/treatment prediction method using characteristic constituent units in the test analysis/diagnosis/treatment prediction apparatus as steps. Further, one embodiment of the present disclosure can be a computer program causing a computer to execute each characteristic step in the test analysis/diagnosis/treatment prediction method. Further, one embodiment of the present disclosure can be a computer readable non-transient recording medium on which such a computer program is recorded.

In each of the embodiments described above, each constituent element can be comprised of a dedicated hardware or materialized by executing a software program that is suited to each constituent element. Each constituent element can be materialized by a program execution unit such as a CPU or a processor reading out and executing a software program recorded on a recording medium such as a hard disk or semiconductor memory. In this regard, software materializing the pain estimation apparatuses of each of the embodiments described above can be a program described above herein.

As used herein, “or” is used when “at least one or more” of the listed matters in the sentence can be employed. When explicitly described herein as “within the range of two values”, the range also includes the two values themselves.

Reference literatures such as scientific literatures, patents, and patent applications cited herein are incorporated herein by reference to the same extent that the entirety of each document is specifically described.

The present disclosure has been described while showing preferred embodiments to facilitate understanding. The present disclosure is described hereinafter based on Examples. The above descriptions and the following Examples are not provided to limit the present disclosure, but for the sole purpose of exemplification. Thus, the scope of the present disclosure is not limited to the embodiments or the Examples specifically described herein and is limited only by the scope of claims.

### EXAMPLES

Examples are described hereinafter.

### (Example 1) Analysis by AI Using DNA and RNA

This Example demonstrates AI analysis through the steps of:

(1) data acquisition (transcriptome data, genomic sequence data, mutation data, genome epigenetics data, miRNA expression data, or RNA methylation data);
(2) image formation;
(3) learning of an image with a machine equipped with both a GPU and CPU; and
(4) prediction of sensitivity to an anticancer agent using another image.

The learning step of (3) can be implemented on a program to detect the number of GPUs, GPU onboard memory, number of CPUs, and memory for CPU for divided learning of an image and prediction integration.

### (Example 1-1) Preprocessing

(Data Acquisition)

Comprehensive analysis data for the following cell lines was acquired:

TABLE 1-1

| | | | | | | | | | |
|---|---|---|---|---|---|---|---|---|---|
| 201T | 22RV1 | 23132-87 | 42-MG-BA | 451Lu | 5637 | 639-V | 647-V | 697 | 769-P |
| 786-0 | 8-MG- | 8305C | 8505C | A101D | A172 | A204 | A2058 | A258 | A2780 |
| A3-KAW | A375 | A388 | A4-Fuk | A427 | A431 | A498 | A549 | A678 | A704 |
| ABC-1 | ACHN | ACN | AGS | ALL-PO | ALL-SIL | AM-38 | AMO-1 | AN8-CA | ARH-77 |
| ASH-3 | ATN-1 | AU565 | AsPC-1 | BALL-1 | BB30-HNC | BB49-HNC | BB65-RCC | BC-1 | BC-2 |
| BC-3 | BCPAP | BE-13 | BE2- | BEN | BFTC- | BFTC- | BHT-101 | BHY | BICR10 |
| BICR22 | BICR31 | BICR78 | BL-41 | BL-70 | BOKU | BPH-1 | BT-20 | BT-474 | BT-483 |
| BT-549 | BV-173 | Becker | BxPC-3 | C-33-A | C-4-I | C2BBe1 | C32 | C3A | CA46 |
| CADO-ES1 | CAKI-1 | CAL-120 | CAL-12T | CAL-148 | CAL-27 | CAL-29 | CAL-33 | CAL-89 | CAL-51 |
| CAL-54 | CAL-62 | CAL-72 | CAL-78 | CAL-85-1 | CAMA-1 | CAPAN-1 | CAS-1 | CCF-STTG1 | CCK-81 |
| CCRF-CEM | CESS | CFPAC-1 | CGTH-W-1 | CHL-1 | CHP-126 | CHP-184 | CHP-212 | CHSA0011 | CHSA0108 |
| CHSA8926 | CL-11 | CL-34 | CL-40 | CMK | CML-T1 | COLO-205 | COLO-320- | COLO-668 | COLO-678 |
| | | | | | | | HSR | | |
| COLO-679 | COLO-680N | COLO-684 | COLO-741 | COLO-783 | COLO-792 | COLO-800 | COLO-824 | COLO-829 | COR-L105 |
| COR-L23 | COR-L279 | COR-L308 | COR-L311 | COR-L32 | COR-L321 | COR-L88 | COR-L95 | CP50-MEL-B | CP66-MEL |
| CP67-MEL | CPC-N | CRO-AP2 | CRO-AP8 | CS1 | CTB-1 | CTV-1 | CW-2 | Ca-Ski | Ca9-22 |
| CaR-1 | Calu-1 | Calu-3 | Calu-6 | Caov-3 | Caov-4 | Capan-2 | ChaGo-K-1 | D-247MG | D-263MG |
| D-283MED | D-836MG | D-392MG | D-428MG | D-502MG | D-542MG | D-566MG | DAN-G | DB | DBTRG- |
| | | | | | | | | | 05MG |
| DEL | DG-75 | DIFI | DJM-1 | DK-MG | DMS-114 | DMS-153 | DMS-273 | DMS-53 | DMS-79 |
| DND-41 | DOHH-2 | DOK | DOV18 | DSH1 | DU-145 | DU-4475 | DV-90 | Daoy | Daudi |
| Detroit562 | DoTc2-4510 | EB-3 | EB2 | EBC-1 | EC-GI-10 | ECC10 | ECC12 | ECC4 | EFE-184 |
| EFM-19 | EFM-192A | EFO-21 | EFO-27 | EGI-1 | EHEB | EJM | EKVX | EM-2 | EMC-BAC-1 |
| EMC-BAC-2 | EN | EPLC-272H | ES-2 | ES1 | ES3 | ES4 | ES5 | ES6 | ES7 |
| ES8 | ESO26 | ESO51 | ESS-1 | ETK-1 | EVSA-T | EW-1 | EW-11 | EW-12 | EW-13 |
| EW-16 | EW-18 | EW-22 | EW-24 | EW-8 | EW-7 | EW7476 | EoL-1- | FADU | FLO-1 |
| FTC-133 | FU-OV-1 | FU97 | Farage | G-292-Clone- | G-361 | G-401 | G-402 | G-MEL | GA-10 |
| | | | | A141B1 | | | | | |
| GAK | GAMG | GB-1 | GCIY | GCT | GDM-1 | GI-1 | GI-ME-N | GMS-10 | GOTO |
| GP5d | GR-ST | GRANTA- | GT3TKB | H-EMC-SS | H2369 | H2373 | H2461 | H2591 | H2595 |
| | | 519 | | | | | | | |
| H2596 | H2722 | H2731 | H2795 | H2803 | H2804 | H2810 | H2818 | H2869 | H290 |
| H3118 | H3255 | H4 | H513 | H9 | HA7- | HAL-01 | HARA | HC-1 | HCC-15 |
| HCC-38 | HCC-366 | HCC-44 | HCC-56 | HCC-78 | HCC-827 | HCC114 | HCC118 | HCC189 | HCC141 |
| HCC142 | HCC150 | HCC156 | HCC159 | HCC180 | HCC193 | HCC195 | HCC202 | HCC215 | HCC221 |
| HCC299 | HCC38 | HCC70 | HCE-4 | HCT-116 | HCT-15 | HD-MY- | HDLM-2 | HDQ-P1 | HEC-1 |
| HEL | HGC-27 | HH | HL-60 | HLE | HMV-II | HN | HO-1-N- | HO-1-n-1 | HOP-62 |
| HOP-92 | HOS | HPAC | HPAF-II | HSC-2 | HSC-3 | HSC-39 | HSC-4 | HT | HT-1080 |
| HT-115 | HT-1197 | HT-1376 | HT-144 | HT-29 | HT-3 | HT55 | HTC-C3 | HUH-6-clone5 | HUTU-80 |
| HeLa | Hep3B2-1-7 | Hey | Hs-445 | Hs-578-T | Hs-633T | Hs-683 | Hs-766T | Hs-939-T | Hs-940-T |
| Hs746T | HuCCT1 | HuH-7 | HuO- | HuO9 | HuP-T8 | HuP-T4 | IA-LM | IGR-1 | IGR-37 |
| IGROV-1 | IHH-4 | IM-9 | IM-95 | IMR-5 | IOSE- | IOSE-397 | IOSE- | IOSE-75- | IPC-298 |
| | | | | | 364(—) | | 523(—) | 16SV40 | |

TABLE 1-2

| | | | | | | | | | |
|---|---|---|---|---|---|---|---|---|---|
| IST- | IST-MES1 | IST-SL1 | IST-SL2 | Ishikawa | J-RT3-T8-5 | J82 | JAR | JEG-3 | JEKO-1 |
| MEL1 | | | | (Heraklio) | | | | | |
| | | | | 02ER- | | | | | |
| JHH-1 | JHH-2 | JHH-4 | JHH-6 | JHH-7 | JHOS-2 | JHOS-3 | JHOS-4 | JHU-011 | JHU-013 |
| JHU-019 | JHU-022 | JHU-028 | JHU-029 | JIMT-1 | JJN-3 | JM1 | JSC-1 | JURL- | JVM-2 |
| | | | | | | | | MK1 | |
| JVM-3 | JiyoyeP- | Jurkat | K-562 | K052 | K1 | K19 | K2 | K4 | K5 |
| | 2003 | | | | | | | | |
| K8 | KALS-1 | KARPAS- | KARPAS- | KARPAS-299 | KARPAS- | KARPAS- | KARPAS- | KASUMI- | KATO1II |
| | | 1106P | 231 | | 422 | 45 | 620 | 1 | |
| KCL-22 | KE-37 | KELLY | KG-1 | KG-1-C | KGN | KINGS-1 | KLE | KM-H2 | KM12 |
| KMH-2 | KMOE-2 | KMRC-1 | KMRC-20 | KMS-11 | KMS-12-BM | KNS-42 | KNS-62 | KNS-81- | KON |
| | | | | | | | | FD | |
| KOPN-8 | KOSC-2 | KP-1N | KP-2 | KP-3 | KP-4 | KP-N-RT- | KP-N-YN | KP-N-YS | KS-1 |
| | | | | | | BM-1 | | | |
| KU-19-19 | KU812 | KURAMOCHI | KY821 | KYAE-1 | KYM-1 | KYSE- | KYSE- | KYSE- | KYSE- |
| | | | | | | 140 | 150 | 180 | 220 |
| KYSE- | KYSE-30 | KYSE-410 | KYSE- | KYSE-50 | KYSE-510 | KYSE- | KYSE-70 | Kasumi-3 | L-1236 |
| 270 | | | 450 | | | 520 | | | |
| L-363 | L-428 | L-540 | LAMA-84 | LAN-6 | LB1047- | LB2241- | LB2518- | LB373- | LB647- |
| | | | | | RCC | RCC | MEL | MEL-D | SCLC |
| LB771- | LB881- | LB996-RCC | LC-1-q | LC-1F | LC-2-ad | LC4-1 | LCLC- | LCLC- | LIM1215 |
| HNC | BLC | | | | | | 108H | 97TM1 | |
| LK-2 | LN-18 | LN-229 | LN-405 | LNCaP-Clone- | LNZTA3WT4 | LOU- | LOUCY | LOXIMVI | LP-1 |
| | | | | FGC | | NH91 | | | |
| LS-1034 | LS-123 | LS-180 | LS-411N | LS-513 | LU-134- | LU-135 | LU-139 | LU-165 | LU-65 |
| LU-99A | LXF-289 | LoVo | M059J | M14 | MB157 | MC-1010 | MC-CAR | MC-IXC | MC116 |
| MCAS | MCC13 | MCC26 | MCF7 | MDA-MB-134- | MDA-MB- | MDA- | MDA- | MDA- | MDA- |
| | | | | | 157 | MB-175- | MB-231 | MB-330 | MB-361 |
| MDA- | MDA- | MDA-MB-453 | MDA- | MDST8 | ME-1 | ME-180 | MEC-1 | MEG-01 | MEL-HO |
| MB-415 | MB-436 | | MB-468 | | | | | | |
| MEL- | MES-SA | MFE-280 | MFE-296 | MFE-319 | MFH-ino | MFM-228 | MG-68 | MHH- | MHH-ES- |
| JUSO | | | | | | | | CALL-2 | 1 |
| MHH-NB- | MHH- | MIA-PaCa-2 | MKL-1- | MKL-2 | MKN1 | MKN28 | MKN45 | MKN7 | ML-1 |
| 11 | PREB-1 | | subclone-2 | | | | | | |
| ML-2 | MLMA | MM1S | MMAC- | MN-60 | MOG-G-CCM | MOG-G- | MOLM- | MOLM-16 | MOLP-8 |
| | | | SF | | | UVW | 13 | | |
| MOLT-13 | MOLT-16 | MOLT-4 | MONO- | MPP-89 | MRK-nu-1 | MS-1 | MS751 | MSTO-211H | MV-4-11 |
| | | | MAC-6 | | | | | | |
| MY-M12 | MZ1-PC | MZ2-MEL | MZ7-mel | Mewo | Mo-T | NALM-6 | NAMALWA | NB(TU)1-10 | NB1 |
| NB10 | NB12 | NB13 | NB14 | NB17 | NB4 | NB5 | NB6 | NB69 | NB7 |
| NBsusSR | NCC010 | NCC021 | NCI-H1048 | NCI-H1092 | NCI-H1105 | NCI-H1155 | NCI-H1184 | NCI-H128 | NCI-H1299 |
| NCI-H1304 | NCI-H1341 | NCI-H1355 | NCI-H1385 | NCI-H1395 | NCI-H1404 | NCI-H1417 | NCI-H1435 | NCI-H1436 | NCI-H1437 |
| NCI-H146 | NCI-H1522 | NCI-H1563 | NCI-H1568 | NCI-H1573 | NCI-H1581 | NCI-H1618 | NCI-H1623 | NCI-H1648 | NCI-H1650 |
| NCI-H1651 | NCI-H1666 | NCI-H1688 | NCI-H1693 | NCI-H1694 | NCI-H1703 | NCI-H1734 | NCI-H1765 | NCI-H1770 | NCI-H1781 |
| NCI-H1792 | NCI-H1793 | NCI-H1836 | NCI-H1838 | NCI-H1869 | NCI-H187 | NCI-H1876 | NCI-H1915 | NCI-H1926 | NCI-H1944 |
| NCI-H196 | NCI-H1963 | NCI-H1975 | NCI-H1993 | NCI-H2009 | NCI-H2023 | NCI-H2029 | NCI-H2080 | NCI-H2052 | NCI-H2066 |
| NCI-H2081 | NCI-H2085 | NCI-H2087 | NCI-H209 | NCI-H2107 | NCI-H211 | NCI-H2110 | NCI-H2122 | NCI-H2126 | NCI-H2185 |
| NCI-H2141 | NCI-H2170 | NCI-H2171 | NCI-H2172 | NCI-H2196 | NCI-H220 | NCI-H2227 | NCI-H2228 | NCI-H226 | NCI-H2286 |

TABLE 1-3

| | | | | | | | | | |
|---|---|---|---|---|---|---|---|---|---|
| NCI-H2291 | NCI-H23 | NCI-H2342 | NCI-H2347 | NCI- | NCI- | NCI- | NCI-H250 | NCI-H28 | NCI-H292 |
| | | | | H2405 | H2444 | H2452 | | | |
| NCI-H3122 | NCI-H322M | NCI-H345 | NCI-H358 | NCI-H378 | NCI-H441 | NCI-H446 | NCI-H460 | NCI-H508 | NCI-H510A |
| NCI-H520 | NCI-H522 | NCI-H524 | NCI-H526 | NCI-H596 | NCI-H630 | NCI-H64 | NCI-H647 | NCI-H660 | NCI-H661 |
| NCI-H69 | NCI-H716 | NCI-H719 | NCI-H720 | NCI-H727 | NCI-H735 | NCI-H747 | NCI-H748 | NCI-H810 | NCI-H82 |
| NCI-H820 | NCI-H835 | NCI-H838 | NCI-H841 | NCI-H847 | NCI-H865 | NCI-H929 | NCI-N87 | NCI-SNU-1 | NCI-SNU-16 |
| NCI-SNU-5 | NEC8 | NH-12 | NK-92MI | NKM-1 | NMC-G1 | NOMO-1 | NOS-1 | NTERA-S-cl- | NU-DUL-1 |
| | | | | | | | | D1 | |
| NUGC-8 | NUGC-4 | NY | OACM5-1 | OACp4C | OAW-28 | OAW-42 | OC-814 | OCI-AML2 | OCI-AML3 |
| OCI-AML5 | OCI-LY-19 | OCI-LY7 | OCI-M1 | OCUB-M | OCUM-1 | OE19 | OE21 | OE38 | OMC-1 |
| ONS-76 | OPM-2 | OS-RC-2 | OSA-80 | OSC-19 | OSC-20 | OUMS- | OV-17R | OV-56 | OV-7 |
| OV-90 | OVCA42 | OVCA43 | OVCA43 | OVCAR- | OVCAR- | OVCAR- | OVCAR- | OVISE | OVK-18 |
| OVKATE | OVMIU | OVTOKO | P116 | P12- | P30-OHK | P31-FUJ | P32-ISH | P3HR-1 | PA-1 |
| | | | | ICHIKAWA | | | | | |
| PA-TU-8902 | PA-TU- | PANC-02- | PANC-03- | PANC-04-03 | PANC-08- | PANC-10- | PC-14 | PC-3 | PC-3 [JPC- |
| | 8988T | 03 | 27 | | 13 | 05 | | | |
| PCI-15A | PCI-30 | PCI-38 | PCI-4B | PCI-6A | PE-CA-PJ15 | PEO1 | PF-382 | PFSK-1 | PL-21 |
| PL18 | PL4 | PLC-PRF-5 | PSN1 | PWR-1E | QGP-1 | QIMR-WIL | RC-K8 | RCC-AB | RCC-ER |
| RCC-FG2 | RCC-JF | RCC-JW | RCC-MP | RCC10RGB | RCH-ACV | RCM-1 | RD | RD-ES | REH |
| RERF-GC- | RERF-LC- | RERF-LC- | RERF- | RF-48 | RH-1 | RH-18 | RH-41 | RKN | RKO |
| 1B | KJ | MS | LC-Sql | | | | | | |
| RL | RL95-2 | RMG-1 | RO82-W-1 | ROS-50 | RPMI-2650 | RPMI-6666 | RPMI- | RPMI-8226 | RPMI-8402 |
| | | | | | | | 7951 | | |
| RPMI-8866 | RS4-11 | RT-112 | RT4 | RVH-421 | RXF393 | Raji | Ramos-2G6- | S-117 | SAS |
| SAT | SBC-1 | SBC-3 | SBC-5 | SCC-15 | SCC-25 | SCC-3 | SCC-4 | SCC-9 | SCC90 |
| SCH | SCLC-21H | SCaBER | SF126 | SF268 | SF295 | SF589 | SH-4 | SHP-77 | SIG-M5 |
| SIMA | SISO | SJRH30 | SJSA-1 | SK-CO-1 | SK-ES-1 | SK-GT-2 | SK-GT-4 | SK-HEP-1 | SK-LMS-1 |
| SK-LU-1 | SK-MEL-1 | SK-MEL-2 | SK-MEL-24 | SK-MEL-28 | SK-MEL-3 | SK-MEL-30 | SK-MEL-31 | SK-MEL-5 | SK-MES-1 |
| SK-MG-1 | SK-MM-2 | SK-N-AS | SK-N-DZ | SK-N-FI | SK-N-SH | SK-NEP-1 | SK-OV-3 | SK-PN-DW | SK-UT-1 |
| SKG-IIIa | SKM-1 | SKN | SKN-3 | SLVL | SN12C | SNB75 | SNG-M | SNU-1040 | SNU-175 |
| SNU-182 | SNU-387 | SNU-398 | SNU-407 | SNU-423 | SNU-449 | SNU-475 | SNU-61 | SNU-81 | SNU-C1 |
| SNU-C2B | SNU-C5 | SR | ST486 | STS-0421 | SU-DHL-1 | SU-DHL-10 | SU-DHL-16 | SU-DHL-4 | SU-DHL-5 |
| SU-DHL-6 | SU-DHL-8 | SU8686 | SUIT-2 | SUP-B15 | SUP-B8 | SUP-HD1 | SUP-M2 | SUP-T1 | SW1088 |
| SW1116 | SW1271 | SW18 | SW1417 | SW1463 | SW156 | SW1573 | SW1710 | SW1783 | SW1990 |
| SW403 | SW48 | SW620 | SW626 | SW684 | SW756 | SW780 | SW837 | SW872 | SW900 |
| SW948 | SW954 | SW962 | SW982 | Saos-2 | Sarc9871 | Sci-1 | Sot2 | SiHa | T-24 |
| T-T | T47D | T84 | T98G | TALL-1 | TASK1 | TC-71 | TC-YIK | TCCSUP | TE-1 |
| TE-10 | TE-11 | TE-12 | TE-15 | TE-4 | TE-441- | TE-5 | TE-6 | TE-8 | TE-9 |
| TF-1 | TGBC11TKB | TCBC1TKB | TGBC24TKB | TGW | THP-1 | TI-73 | TK | TK10 | TMK-1 |
| TOV-112D | TOV-21G | TT | TT2609-C02 | TUR | TYK-nu | Takigawa | Tera-1 | Toledo | U-118-MG |

TABLE 1-4

| | | | | | | | | | |
|---|---|---|---|---|---|---|---|---|---|
| U-2-OS | U-266 | U-698-M | U-87-MG | U-CH1 | U031 | U251 | UACC-257 | UACC-62 | UACC-812 |
| UACC-893 | UDSCC2 | UISO-MCC-1 | UM-UC-3 | UMC-11 | UWB1.289 | VA-ES-BJ | VAL | VCaP | VM-CUB-1 |
| VMRC-LCD | VMRC-MELG | VMRC-RCW | VMRC-RCZ | WIL2-NS | WM-115 | WM1158 | WM1552C | WM239A | WM278 |
| WM35 | WM793B | WM902B | WSU-DLCL2 | WSU-NHL | YAPC | YH-13 | YKG-1 | YMB-1-E | YT |
| ZR-75-30 | huH-1 | no-10 | no-11 | | | | | | |

Comprehensive analysis data is managed at Genomics of Drug Sensitivity in Cancer (GDSC; https://www.cancerrxgene.org/). The data was acquired for this site. As the data, transcriptome data, genomic sequence data, mutation data, genome epigenetics data, miRNA expression data, and RNA methylation data in each cell line were acquired. Expression data was downloaded directly from EMBL-EBI ArrayExpress, E-MTAB-3610 Transcriptional Profiling of 1,000 human cancer cell lines (https://www.ebi.ac.uk/arrayexpress/experiments/E-MTAB-3610/), and mutation data and sensitivity data were downloaded directly from GDSC (https://www.cancerrxgene.org/downloads). For each cell line, information on resistance to 5-FU was acquired.

(Equipment Used for Image Formation)

The following equipment was used for image formation. As is apparent to those skilled in the art, it is understood that any equipment equivalent thereto can be used in the same manner.

Windows® 7, Core i7-4810MQ 2.80 GHz, macOS X10.13.6 3.5 GHz 6-Core Intel Xeon E5, and CentOS 6.4 Intel Xeon E5-2697 v2@2.70 GHz were concurrently used as the equipment. However, a computer for image formation is not particularly limited as long as an operating environment that allows use of the latest version of R or ifort is provided. The amount of computation is an amount that is sufficiently computed with any one of the cores. Parallelization only has an effect on reduction of time. For the software, a self-made program using R and Fortran was used for processing.

(Method of Image Formation)

For image formation, an expression unit was assigned to a two-dimensional numerical value matrix arranged in the vertical and horizontal directions. Specifically, all genes and miRNAs registered in Ensemble were each used as an expression unit. One pixel is assigned to one element of the numerical value matrix. With a rectangular region of 125 pixels (rows) vertically and 2 pixels (column) horizontally (250 pixel unit) as one unit, a plurality of the horizontally adjacent unit regions were assigned in accordance with the length of expression unit. Each pixel is set to one of 256 levels of colors [brightness for monochrome] (0 to 255).

The amount of expression was found from the data acquired above for each expression unit. The frequency of each gene or exon appearing within a transcriptome was counted and standardized by the total read length of the transcriptome as the amount of expression of each exon. The number of reads from mapping the amount of expression of each miRNA to each miRNA in miRNA sequencing data was standardized by the total read length as the amount of expression of each miRNA. The amount of expression was normalized and grouped into 150 levels. The left side column of a 250 pixel unit in each expression unit was set to one of colors with a concentration of 1 to 150 associated with the amount of expression.

Sequence data was found from the data acquired above for each expression unit. Information on details of mutations and the portion where the mutations are located in the genome and in each cell line was acquired from the reference sequences for a partial sequence encoding miRNA and each exon and genome data acquired above. Information on each mutation was reflected in a region assigned to each expression unit. Each pixel of a row in each region corresponds to a position in a sequence in an expression unit.

If there was a base substitution, relative to the reference sequence, in a partial sequence in a genome encoding each miRNA and each gene or exon, a pixel on the left side of a row in a 250 pixel unit corresponding to each substitution position was set to a color of adenine (200), thymine (210), guanine (220), or cytosine (230) in accordance with the base after the mutation.

If there was a base deletion, relative to the reference sequence, in a partial sequence in a genome encoding each miRNA and each gene or exon, a pixel on the left side of a row in a 250 pixel unit corresponding to each substitution position was set to a color of 250 (deletion).

If there was a base insertion, relative to the reference sequence, in a partial sequence in a genome encoding each miRNA and each exon, a pixel on the left side of a row in a 250 pixel unit corresponding to the starting position of each insertion was set to a color of 180 (start of insertion), and pixels, starting from the pixel on the right side of the pixel with the color of 180, were sequentially set, one by one, to a color of adenine (200), thymine (210), guanine (220), or cytosine (230) in accordance with the inserted base sequence.

If an epigenetic modification was detected in a partial sequence in a genome encoding each miRNA and each gene or exon, a pixel on the right side of a row in a 250 pixel unit corresponding to each modification position was set to a color in accordance with the types of modifications described below.

DNA methylation: 186, histone acetylation: 188, histone methylation: 190, histone ubiquitination: 192, histone phosphorylation: 194, histone sumoylation: 196.

When methylation was detected in each RNA, a pixel on the left side of a row in a 250 pixel unit corresponding to each modification position was set to a methylation color in the following manner. For methylation of mRNA, m6A: 235, Am: 236, M6Am: 237, m62Am: 238, I: 240, m5C: 242, Cm: 243, m7G: 245, Gm: 246, m27G: 248, m227G: 249, Um: 251, M3Um: 252. It is understood that this can be adapted to methylation of tRNA, mrRNA, or the like by adding colors (e.g., changed 256 colors, 16 Bit color, or the like).

The steps described above were performed for each expression unit of each cell to generate an image summarizing expression data and sequence data for each cell.

### (Example 1-2) Analysis

(Feature Extraction)

A differentiation parameter is optimized by machine learning using a neural network for image analysis. In doing so, a characteristic portion is extracted by continuation in brightness and saturation from a partial image. The differentiation parameter coefficient is then optimized. A differentiation model using the coefficient is constructed.

(Classification)

Data is classified into groups based on the resulting differentiation model using the differentiation parameter.

### (Example 2) Improved Arrangement on Array

(Correlation Analysis)

The degree of tendency to change in tandem is analyzed for all gene sets using normalized gene expression information in all registered cell lines. In doing so, the Pearson correlation coefficient and Spearman's correlation coefficient, as well as the average numerical value thereof, were computed. Gene names extracted as the top combinations (100 in this Example) with strong correlation were counted.

(Multiple Regression)

It is determined what coefficient can be added to describe a gene using the amount of expression of another gene (normalized value) in the order of genes with highest count in correlation analysis (whether this can be described by linear combination).

(Optimization)

The gene with the highest count from extraction in correlation analysis is arranged in the middle of an array. A correlated set with the target gene is then extracted, and the mean values of the Pearson and Spearman's correlation coefficients are used as interaction coefficients in the gene region to be arranged (125 rows×00 columns). The initial arrangement from the center gene is set to be inversely proportional to the interaction coefficient. The same arrangement is also repeated from the subsequently arranged gene to set the initial arrangement. At subsequent optimization, interaction in gene regions considers an average interaction coefficient like a spring constant. The position is optimized only in the horizontal direction of the initial arrangement. For this reason, a deviation is not allowed between genes in each partial row (125 row unit), but a deviation in the location in contact to the top and bottom of a partial region of a gene to the left or right is acceptable due to a force in accordance with the aforementioned spring constant. An algorithm is used which searches for the optimal arrangement as a result thereof.

### (Example 3) Improving Efficiency of Calculation

(Machine Specification Detection)

For the machine used in machine learning in this Example, a program is created for the Linux® OS. In such a case, the specification of the CPU can be found by using the command cat/proc/cpuinfo.

Similarly, the machine specification can be found using cat/proc/meminfo for memory,

lspci|grepVGA for GPU, and
nvidia-smi when an NVIDIA driver is installed.

(Division of Data)

Since machine learning of images presumes learning using GPU, data is divided while taking into consideration learning data count and verification data count that can be loaded into memory in view of the GPU onboard memory.

(Integration of Data)

Coefficient parameters of each model generated by divided learning are stored in a matrix matching the dimensions of the neural network. A divided parameter matrix is stored in a single matrix. In this regard, a new prediction model using the preliminary parameter as the initial value is constructed.

(Optimization)

The rate of change in the prediction efficiency when a partial parameter of a prediction model using integrated initial parameter is changed is observed. The most stable parameter is found by non-linear optimization. This calculation performs optimization using a CPU by using HDD as a virtual memory and interacting on the fly with the memory.

### (Example 4) Analysis Example

Comprehensive transcriptome data, genomic sequence data, and mutation data were acquired for the target tumor cell lines. A model obtained by the learning described above is applied to predict 5-FU resistance of the tumor cell lines. Information on 5-FU resistance of the tumor cell lines is acquired to verify the validity of the model.

### (Example 4-1) Analysis Example of Anticancer Agent Sensitivity

Comprehensive transcriptome data, genomic sequence data, and mutation data were acquired for tumor cell lines described in (Data acquisition) of Example 1. 20 tumor cell lines including 10 cells lines with particularly high sensitivity to 5-FU (MV-4-11, NOMO-1, OCI-AML2, PSN1, RPMI-6666, SIG-M5, SLVL, SR, SUP, and YT) and 10 cells lines with particularly low sensitivity to 5-FU (CAS-1, FU-OV-1, HCC1143, NCI-H1693, NCI-H2291, OVKATE, Saos-2, SKG-IIIa, SW684, and SW111) were used as training data.

The modification described in Example 2 was applied to the procedure described in (Image formation method) of Example 1 to form the data described above into an image.

For the images, machine learning was performed on the correlation between the image and anticancer agent sensitivity in accordance with the procedure described in (Feature extraction) and (Classification) described in Example 1 and (Division of data) described in Example 3. Specifically, the generated image was divided into 16×16, and a differentiation parameter was optimized by machine learning using a neural network for image analysis for each region, and a model was created for each region.

A new differentiation formula (for the entire image before dividing) that integrates differentiation formulas from parameters found in learning for each region was created, based on the differentiation formulas. To do so, a model for predicting sensitivity to an anticancer agent from the entire image was generated by optimizing the whole using a CPU, with the parameter of each partial learning as the initial value.

The prediction accuracy of the generated model was tested each time learning was repeated, with one run of learning of data for all 20 types of cell lines counted as one epoch. The percentage of correct answers in predicting 5-FU sensitivity of the cell lines was studied based on an image generated in the same manner from a cell line that is different from those used in learning. FIG. 9 shows the relationship between the number of epochs and percentage of correct answer. Constructed differentiation models were able to differentiate at a 100% accuracy with respect to cell lines using non-learned image (FIG. 9).

The same test was performed on CDDP (cisplatin) sensitivity), which was also able to differentiate at 100% accuracy.

### (Example 4-2) Change in Learning Efficiency by Data Type Used in Image Formation

Training data for tumor cell lines was acquired in accordance with the method described in Example 4-1. In addition to images formed from both DNA mutation data and RNA expression amount data described in Example 4-1, an image formed in the same manner from information on only DNA mutation data and image formed in the same manner from information on only RNA expression amount data were created.

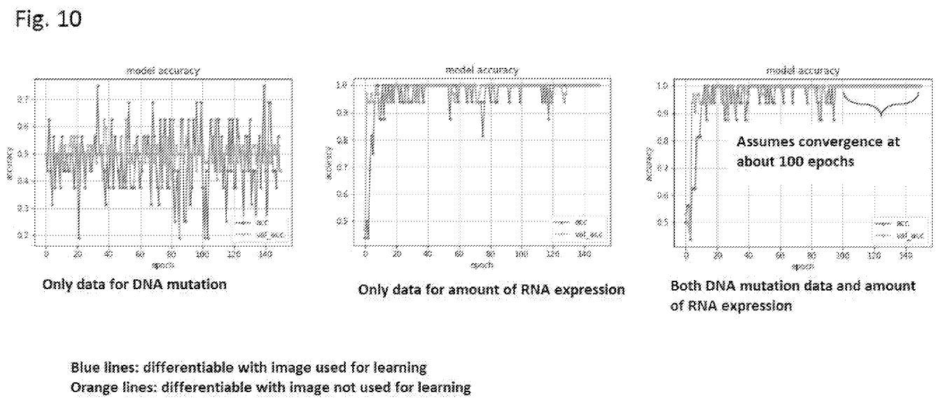

Each image was subjected to the same learning as Example 4-1 to test the accuracy of the models generated for each epoch. As the model accuracy, differentiability with image used in learning and differentiability with an image not used in learning were studied. FIG. 10 shows the results.

It is understood that it is difficult to generate a model that can differentiate anticancer agent sensitivity from only DNA mutation data. When using only expression amount data, it is understood that a differentiable model can be generated by repeating learning. However, when using both data, it is understood that accuracy converges to 100% (1.0 in the graph of FIG. 10) at about 100 epochs, so that learning can be more efficient. When the standard deviation of the percentages of correct answers when using only expression amount data is compared to those when using both data, the value of standard deviation reached at 100 epochs when using only expression amount data was reached at 58 epochs when using both data. In view of the above, the number of learning reaching the same accuracy can be reduced on average by about 40% when using both data.

### (Example 4-3) Difference in Convergence by Divided Regions

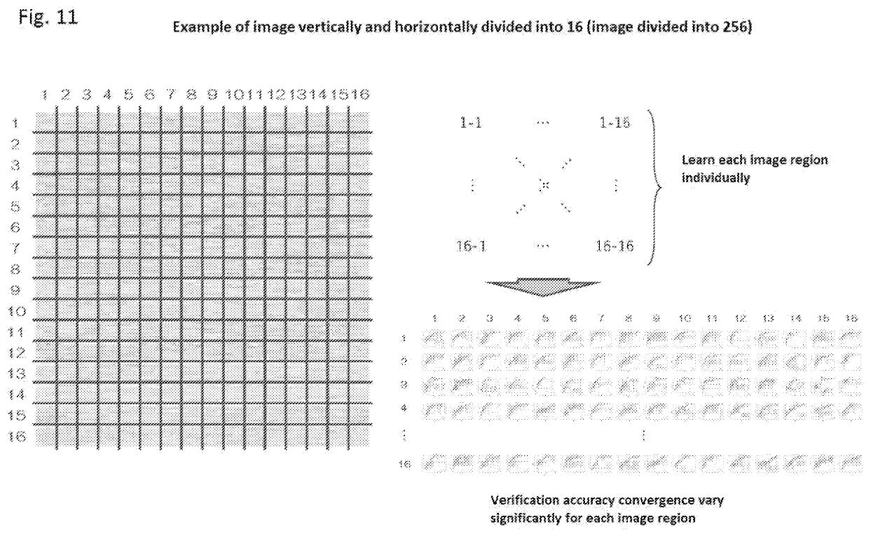

As described in Example 4-1, a generated image was divided into 16×16, a differentiation parameter was optimized by machine learning using a neural network for image analysis for each region, and a model was generated for each region. With the division described above, information on about 100 to 200 genes is stored for each region. Convergence of verification accuracy for each epoch was tested for models for each region (FIG. 11).

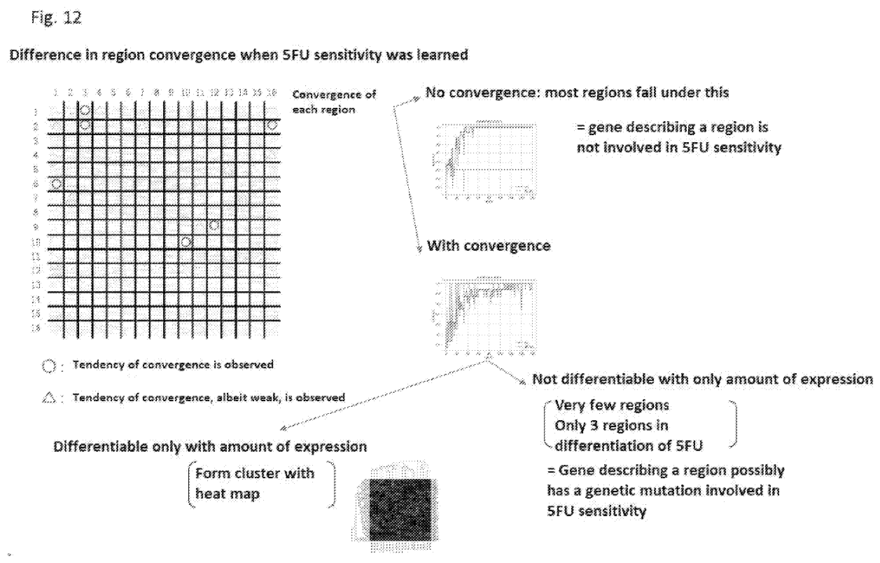

When region convergence from learning 5FU sensitivity was studied, it was found that most regions fell under a region without convergence (percentage of correct answers does not converge to 1 even when the number of epochs is increased), but a model with convergence was generated in some regions (FIG. 12). It is understood that models generated in these regions themselves can be utilized in prediction of anticancer agent sensitivity. It is understood that a model for predicting sensitivity to an anticancer agent from the entire image can be generated by integrating and learning data by focusing on these regions with convergence.

Furthermore, each of the regions with a tendency for convergence was studied as to whether it is capable of differentiation with information on the amount of expression. Specifically, cluster analysis was performed on the amount of expression of genes in a region with a tendency for convergence in each cell line to study whether the amount is correlated with sensitivity to an anticancer agent.

Cluster analysis was performed based on each amount of expression of genes in a divided region. Since there are two target differentiation groups each having the same number, each of the individuals arranged in accordance with similarity was separated in the middle, and the ratio of identity within each separated group was computed. A ratio of identify of each individual of 100% indicates that each can be completely separated only with expression information, and 50% indicates random division and unable to separate only with expression information. This Example deemed individuals to be differentiable with only the amount of expression at 1 or 2 differences among 10, i.e., 80 to 90% or greater.

The majority of regions with a tendency of convergence was capable of differentiating anticancer agent sensitivity with only information on the amount of expression, but sensitivity to an anticancer agent could not be differentiated from only the amount of expression for a small number of regions among regions with convergence. A gene describing such a region possibly has a genetic mutation involving 5FU sensitivity. In view of the above, it is understood that a model for predicting sensitivity to an anticancer agent from a genetic mutation can be generated. Further, it is understood that a difference in convergence for each region can be applied to a method of identifying a mutation of a gene involving in a certain trait.

A gene region that affects the efficacy of an anticancer agent can be identified by divided learning of an anticancer agent efficacy determining model. Identification of a gene region involved in anticancer agent resistance using whole genome information can possibly elucidate a new correlation between a gene and anticancer agent resistance that has not been found previously, which can lead to the development of a novel comparison diagnosis method for anticancer agents.

This Example studied a prediction model for sensitivity to an anticancer agent, but it is understood that a prediction model can be similarly generated for traits other than sensitivity to an anticancer agent if other traits are used as learning data.

### (Example 5) Example Including Methylation Other than DNA/RNA Expression

Comprehensive transcriptome data, genomic sequence data, mutation data, epigenetic modification data for DNA, and epigenetic modification data for RNA were acquired for a plurality of tumor cell lines. An image is formed as described above with all such information. The image is used to learn the relationship between information on drug resistance of the tumor cell lines and genetic information as described above. A model generated by learning is applied to predict drug resistance of a target cell line. Some or all of the comprehensive transcriptome data, genomic sequence data, mutation data, epigenetic modification data for DNA, and epigenetic modification data for RNA can be acquired from the target cell line for model application.

### (Example 6) Providing Service to Healthcare Service

A new drug is administered to cancer cells. DNA/RNA information obtained therefrom is learned and analyzed with the system described above to predict the mechanism of action of the drug. The predicted mechanism of action can be provided to, for example, a pharmaceutical company.

Results of responses to an anticancer agent are predicted with the system described above to support drug selection in anticancer agent therapy. The predicted result is provided to, for example, a hospital.

The relationship between genetic information on a plurality of subjects and developed disease is learned with the system described above. From the genetic information on a target subject, information on a disease that the subject can develop can be provided based on a model obtained therefrom.

The relationship between genetic information of a subject with a certain disease and response of the subject to a drug is learned with the system described above. Information on a drug that is considered effective for the target subject can be provided based on a model obtained therefrom.

An application that, upon input of genetic information, transmits the genetic information, receives a result of application to the model described above, and displays a desired result, can also be provided. The application may be capable of forming an image of the genetic information.

A medical support system for predicting the optimal anticancer agent for a cancer patient from sequence image data of the patient is developed and provided. It is understood that such a system contributes to materialization of a truly individualized medicine. A system for selecting the optimal anticancer agent is constructed to provide commissioned testing and/or diagnostic assistance service on the cloud or the like upon a request from a medical institution or testing agency. Data accumulation is also envisioned. Application to therapy of diseases other than anticancer agents, prediction of an effect, side effect, etc. in the development of a new drug by a pharmaceutical company, sequence data analysis service in fundamental research, and the like are provided. A platform for machine learning of genomic information is provided.

(Note)

As disclosed above, the present disclosure has been exemplified by the use of its preferred embodiments. However, it is understood that the scope of the present disclosure should be interpreted based solely on the Claims. It is also understood that any patent, patent application, and references cited herein should be incorporated herein by reference in the same manner as the contents are specifically described herein.

The present application claims priority to Japanese Patent Application No. 2018-247959 (filed on Dec. 28, 2018). The entire content thereof is incorporated herein by reference in its entirety for any purpose.

### INDUSTRIAL APPLICABILITY

The present disclosure can be used in the field where prediction of traits of individuals is useful, particularly the medical field. The present disclosure is useful in prediction of a tendency of development of a disease in advance as well as, for example, determination of suitable treatment or the like.

### REFERENCE SIGNS LIST

101: system
102: storage unit
103: learning unit
104: calculation unit
105: image formation unit
106: display unit
107: acquisition unit
108: database
109: measurement unit

1. A system for predicting trait information on an individual, comprising:
a storage unit for storing genetic information on a plurality of individuals and trait information on the plurality of individuals, the genetic information containing at least two types of information;
a learning unit configured to learn a relationship between genetic information and trait information from the genetic information on the plurality of individuals and the trait information on the plurality of individuals; and
a calculation unit for predicting trait information on an individual from genetic information on the individual based on the relationship between the genetic information and the trait information.

2. The system of claim 1, wherein the learning unit is configured to learn after forming an image of the genetic information on the plurality of individuals.

3. The system of claim 1, wherein the learning unit is configured to divide the genetic information on the plurality of individuals, learn relationships between partial genetic information and trait information, and integrate relationships between a plurality of pieces of partial genetic information and trait information to learn the relationship between the genetic information and the trait information.

4. The system of claim 1, wherein the genetic information is selected from the group consisting of sequence information expression information, and modification information on a genetic factor.

5. A method of forming an image of sequence data for a genetic factor population comprising a plurality of genetic factors and expression data for a genetic factor population comprising a plurality of genetic factors, comprising the step of:
generating image data for storing the sequence data for the genetic factor population and the expression data for the genetic factor population, the image data having a plurality of pixels, each of which comprising position information and color information.

6. The method of claim 5, wherein each of the plurality of genetic factors is associated with a region in the image data, the step of generating the image data comprising the step of:
converting an amount of expression of the genetic factor into color information in a certain region within a region associated with the genetic factor and/or information on an area of a region having a certain color in the region.

7. The method of claim 5,
wherein the step comprises associating each of the plurality of genetic factors with a region in the image data, and regions associated with each genetic factor are arranged so that those with a high correlation weighting of each genetic factor are in proximity.

8. The system of claim 2, wherein the learning unit is configured to perform the formation of an image of the genetic information on the plurality of individuals by forming an image of sequence data for a genetic factor population comprising a plurality of genetic factors and expression data for a genetic factor population comprising a plurality of genetic factors, by at least generating image data for storing the sequence data for the genetic factor population and the expression data for the genetic factor population, the image data having a plurality of pixels, each of which comprising position information and color information is configured to be performed by the image formation method of any of claims 5 to 7.

9. (canceled)

10. The system of claim 2, wherein the learning unit is configured to use data with the data structure of image data representing sequence information on a genetic factor population comprising a plurality of genetic factors and expression information on a genetic factor population comprising a plurality of genetic factors in learning, wherein:
the image data has a plurality of regions associated with the plurality of genetic factors;
each position in a sequence of a genetic factor is associated with a position within the regions associated with the genetic factor;
information on a substitution, a deletion, and/or an insertion at each position in the sequence of the genetic factor is stored as color information at a position associated with the position; and
expression data for the genetic factor is stored as color information at a certain region in the regions, and/or information on an area of a region having a certain color in the regions.

11. The system of claim 3, wherein the learning unit is configured to learn the relationship between the genetic information and the trait information by a method for creating a model for predicting a relationship between an image and information associated with the image, comprising the steps of:
providing a set of a plurality of images and a plurality of pieces of information associated with the plurality of images;
obtaining a plurality of divided learning data by dividing the plurality of images and learning a relationship between a portion of the plurality of images and information associated with the images; and
integrating the plurality of divided learning data to generate a model for predicting the relationship between the image and the information associated with the image.

12. The method system of claim 11, wherein the step of obtaining a plurality of divided learning data verifies an ability to differentiate each divided learning data, selects divided learning data with an ability to differentiate, and subjects the data to integration.

13. (canceled)

14. The system of claim 1,
wherein the learning unit is configured to divide an image generated by forming an image of the genetic information on the plurality of individuals, learn a relationship between each region of the image and trait information, select a region where a model with an ability to differentiate trait information can be generated from each region, and generate a model for predicting trait information from each region on the image.

15. The system of claim 1,
wherein the learning unit is configured to divide an image generated by forming an image of the genetic information on the plurality of individuals, learn a relationship between each region of the image and trait information, select a region where a model with an ability to differentiate trait information can be generated from each region, determine whether trait information can be predicted based on expression information in each region, and identify a gene having a mutation that is correlated with trait information from a gene in a region where trait information cannot be predicted based on expression information, and
the calculation unit is configured to predict the trait information on the individual based on information on the gene having a mutation that is correlated with the trait information.

16. A non-transitory computer-readable storage medium having computer-executable instructions stored thereon that, when executed by at least one computer processor, cause a method for predicting trait information on an individual to be executed, the method comprising:
an information providing step for providing genetic information on a plurality of individuals and trait information on the plurality of individuals, the genetic information containing at least two types of information;
a learning step for learning a relationship between genetic information and trait information from the genetic information on the plurality of individuals and the trait information on the plurality of individuals; and
a predicting step for predicting trait information on an individual from genetic information on the individual based on the relationship between the genetic information and the trait information.
